(19) United States
(12) Patent Application Publication
(10) Pub. No.: US 2004/0044320 A1
(43) Pub. Date: Mar. 4, 2004

(54) Composites having controlled friction angles and cohesion values

(76) Inventors: Arvinder Pal Singh Kainth, Neenah, WI (US); Richard Norris Dodge II, Appleton, WI (US); Joseph Raymond Feldkamp, Appleton, WI (US); Estelle Anne Ostgard, Appleton, WI (US)

Correspondence Address: KIMBERLY-CLARK WORLDWIDE, INC., 401 NORTH LAKE STREET, NEENAH, WI, 54956

(21) Appl. No.: 10/460,985

(22) Filed: Jun. 13, 2003

Related U.S. Application Data
(60) Provisional application No. 60/406,426, filed on Aug. 27, 2002.

(51) Int. Cl. A61F 13/15; A61F 13/20

(52) U.S. Cl. 604/367; 604/385.01

(57) ABSTRACT

The present invention relates to composites having controlled composite-bed friction angles and/or controlled composite-bed cohesion values. Controlling these composite-bed properties may allow control of the swelling of ingredients in the composite, such as a superabsorbent material; and/or the absorbency, resiliency, and porosity of the absorbent composite. Composites having controlled composite-bed friction angles and/or controlled composite-bed cohesion values may be obtained by employing: superabsorbent material having controlled gel-bed friction angles and/or controlled gel-bed cohesion values; fiber having controlled fiber-bed friction angles and/or controlled fiber-bed cohesion values; or some combination thereof.

### BACKGROUND

People rely on absorbent articles in their daily lives.

Absorbent articles, including adult incontinence articles, feminine care articles, and diapers, are generally manufactured by combining a substantially liquid-permeable topsheet; a substantially liquid-impermeable backsheet attached to the topsheet; and an absorbent core located between the topsheet and the backsheet. When the article is worn, the liquid-permeable topsheet is positioned next to the body of the wearer. The topsheet allows passage of bodily fluids into the absorbent core. The liquid-impermeable backsheet helps prevent leakage of fluids held in the absorbent core. The absorbent core is designed to have desirable physical properties, e.g. a high absorbent capacity and high absorption rate, so that bodily fluids may be transported from the skin of the wearer into the disposable absorbent article.

The present invention relates to absorbent composites, such as an absorbent core comprising fiber and superabsorbent material. More specifically, the present invention pertains to absorbent composites having a modified composite-bed friction angle and/or composite-bed cohesion measured in a composite bed of the ingredients used to make the composite (e.g., a bed of particulate superabsorbent material and fibers arranged in the form of a fibrous matrix to contain the superabsorbent material) and absorbent articles incorporating such absorbent composites. Both the composite-bed friction angle and composite-bed cohesion of a composite of the present invention are controllable and follow a predetermined pattern. Controlling the composite-bed friction angle and composite-bed cohesion of the composite may allow control of phenomena including, but not limited to: the swelling of any superabsorbent material employed in the absorbent composite; stresses experienced by the superabsorbent material and/or other ingredients (e.g., fibers) in an absorbent composite; the permeability of an absorbent composite containing the fiber and superabsorbent material; and/or, the absorbency, resiliency, and porosity of the absorbent composite (it should be noted that, in some cases, only one of these properties—i.e., composite-bed friction angle or composite-bed cohesion—need be controlled). The present invention relates to absorbent composites comprising ingredients, such as fibers and superabsorbents, that result in controlled composite-bed friction angle and composite-bed cohesion values.

For example, the present invention relates to absorbent composites employing fiber treated to manipulate fiber-bed friction angle and/or fiber-bed cohesion; or new fibers having the desired fiber-bed friction angle and/or fiber-bed cohesion characteristics. U.S. Provisional Patent Application Serial No. 60/399,788, entitled “Fiber Having Controlled Fiber-Bed Friction Angles And/Or Cohesion Values, And Composites Made From Same,” filed on Jul. 30, 2002, which discloses such fibers, is hereby incorporated by reference in its entirety in a manner consistent herewith. The present invention also relates to selection of, and treatments for, superabsorbent materials having controlled gel-bed friction angles and/or controlled gel-bed cohesions, including novel superabsorbent materials disclosed in three co-pending applications: U.S. Provisional Patent Application Serial No. 60/399,877, entitled “Superabsorbent Materials Having Low, Controlled Gel-Bed Friction Angles and Composites Made From The Same,” filed on Jul. 30, 2002; U.S. Provisional Patent Application Serial No. 60/399,794, entitled “Superabsorbent Materials Having High, Controlled Gel-Bed Friction Angles and Composites Made From The Same,” also filed on Jul. 30, 2002; and, U.S. Provisional Patent Application Serial No. 60/406,526, entitled “Superabsorbent Materials Having Controlled Gel-Bed Friction Angles and Cohesion Values and Composites Made From Same,” filed on Aug. 27, 2002. These three co-pending applications are incorporated by reference in their entirety in a manner consistent herewith.

The preceding paragraph uses different terms to refer to friction angle and cohesion, depending on whether these properties are measured on an absorbent composite bed, i.e., a bed of two or more ingredients, which, for purposes of this application, will generally be fiber and superabsorbent material; a fiber bed, i.e., a bed of fibers; or a gel-bed, i.e., a bed of swollen superabsorbent material. One method of preparing an absorbent composite having a desired composite-bed friction angle and/or composite-bed cohesion is to prepare an absorbent composite comprising: a superabsorbent material having a desired gel-bed friction angle and/or gel-bed cohesion; a fiber having a desired fiber-bed friction angle and/or fiber-bed cohesion; or, some combination thereof. This is discussed in more detail below, and in the co-pending applications referenced above.

Absorbent composites used in absorbent articles typically consist of an absorbent material, such as a superabsorbent material, mixed with an absorbent composite matrix containing natural and/or synthetic fibers. As fluids enter the absorbent composite, the superabsorbent material swells as it absorbs the fluids. The superabsorbent material contacts the surrounding matrix components and possibly other superabsorbent material as it swells. The full swelling capacity of the superabsorbent material may be reduced due to stresses acting on the superabsorbent materials (e.g., stresses imposed by the matrix on superabsorbent material; external stresses acting on the absorbent composite that comprises a matrix and superabsorbent material, including, for example, stresses imposed on an absorbent composite by a wearer during use; stresses imposed by one portion of the superabsorbent material on another portion of the superabsorbent material, whether directly or indirectly; etc.). Furthermore, stresses acting on an absorbent composite comprising the superabsorbent material may act to reduce interstitial pore volume, i.e., space between superabsorbent material, fibers, other ingredients, or some combination thereof (without being bound to a particular analogy, and for purposes of explanation only, think of a force acting on some unit area of a sponge-like material with pores, with the force per unit area—i.e., stress—acting to reduce the thickness of the sponge-like material, and, therefore, the volume of the pores).

The ability of a material, such as superabsorbent particles, to rearrange within an absorbent composite at any given normal load or stress corresponds to a situation where the shear stress exceeds the shear stress at failure (“τ_{ff}”). The shear stress at failure (“τ_{ff}”), equals the sum of two contributions: a cohesion contribution (“c”), and a friction-angle contribution (“σ_{nff} (tan φ)”). This concept is defined mathematically as τ_{ff}=c+σ_{nff} (tan φ), and is defined in more detail below, both in the section entitled “Overview of Continuum Mechanics, Mohr Circles, and Mohr-Coulomb Failure Theory,” and in the section entitled “Detailed Description of Representative Embodiments” (this relationship, and the attendant discussion, applies generally to any material, including a composite bed, gel bed, or fiber bed). In general terms, the value of the shear stress at failure (“σ_{ff}”) relates to the ability of a material to rearrange. By seeking to reduce the cohesion contribution, the friction-angle contribution, or both, shear stress at failure is reduced, meaning that particles are able to move past one another more readily (i.e., at a lower, applied normal load). As discussed in this application, this is desirable when seeking to minimize phenomena such as the pore-size reduction that may accompany the build up of stress.

By seeking to increase the cohesion contribution, the friction-angle contribution, or both, shear stress at failure is increased, meaning that particles are less able to move past one another. As discussed below, this is desirable when seeking to facilitate, for example, the “locking in” of a desirable pore structure and its corresponding pore size or pore-size distribution.

Note that the cohesion contribution to the calculated shear stress at failure remains constant (for a given material at a given state; e.g., a composite swollen or wetted in a manner described below). Cohesion is the same at zero load or stress—the load or stress at which it is determined experimentally, as discussed below—and at any applied normal load or stress greater than zero. The friction angle contribution, however, is directly proportional to the magnitude of the applied normal stress or load (mathematically, the friction angle contribution equals the tangent of the friction angle —which is constant—multiplied by the magnitude of the applied normal stress or load—which may change). Thus at any applied normal stress or load, the magnitude of the shear stress at failure may be reduced by: (1) decreasing the cohesion of the material being evaluated; (2) decreasing the friction angle of the material being evaluated; or, (3) both. Similarly, the magnitude of the shear stress at failure may be increased by: (1) increasing the cohesion of the material being evaluated; (2) increasing the friction angle of the material; or, (3) both.

As the superabsorbent material swells, it may rearrange into void spaces of the absorbent composite matrix as well as expand readily against the matrix to create additional void space. Also, as the superabsorbent material swells, stresses acting within and/or on the absorbent composite may increase due—at least in part—to expansion of the superabsorbent material, thereby reducing the pore volume between: fibers, superabsorbent material; other ingredients in the absorbent composite; or, some combination there of. The ability to rearrange within the absorbent composite matrix, and the magnitude and extent of the stresses acting within and on the absorbent composite matrix, depend on several factors specifically including a composite-bed friction angle and/or composite-bed cohesion value of the absorbent composite. In addition, as the superabsorbent material moves within the absorbent composite matrix, the superabsorbent material may contact the components, such as fibers and binding materials, of the surrounding matrix. Thus, the frictional and cohesive properties of the composite bed may influence the ability of the superabsorbent material to swell and rearrange or move within the matrix, as well as the magnitude and extent of the stresses acting within and on the composite matrix.

It is often desired that the superabsorbent material be able to rotate and translate within the voids of the absorbent composite to allow the superabsorbent material to swell as close to full swelling capacity as is possible within the matrix. Accordingly, there is a need for an absorbent composite that may facilitate a superabsorbent material more easily rearranging within the void space of the absorbent composite matrix. There is also a need for a way to control the physical mechanics of the composite that: allow a superabsorbent material to rearrange within the absorbent composite matrix; reduce or minimize the stresses acting within or on the absorbent composite or its ingredient(s); and/or, decrease or minimize the reduction in pore volume that may accompany the build up of said stresses.

Also, in cases where absorbent composites have initially high porosity or are already fully swollen, it may be desirable to have a superabsorbent material which does not rearrange within the matrix, and thereby maintains porosity and composite permeability by maintaining the free void spaces within the composite matrix.

### SUMMARY

We have discovered that composites having controlled composite-bed friction angles and/or cohesion values should meet one or more of these needs. Accordingly, the present invention is directed to composites having controlled composite-bed friction angles and/or cohesion values. Absorbent composites of the present invention exhibit controlled composite-bed friction angles and/or cohesion values substantially different than composite-bed friction angles and/or cohesion values of conventional absorbent composites. The absorbent composites of the present invention may be produced, for example, by using non-conventional manufacturing processes to obtain desired composite-bed friction angles and/or cohesion values; by treating fiber, superabsorbent material, or both with additives to increase, decrease, or otherwise control the friction angle and/or cohesion of these individual ingredients; by making or processing fiber, superabsorbent material, or both using non-conventional processes; or, some combination thereof. Composite-bed friction angle and composite-bed cohesion are properties of a composite coming from Mohr-Coulomb failure theory (these properties and this theory are discussed in more detail below).

The absorbent composite of the present invention may comprise a fibrous matrix and a water swellable, water insoluble superabsorbent material in combination with the fibrous matrix in a dosage level of between about 20 and about 40 percent, the absorbent composite having a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals, substantially equal to or less than the first composite-bed friction angle. The first composite-bed friction angle may be about 30 degrees or less.

The absorbent composite of the present invention may comprise a fibrous matrix and a water swellable, water insoluble superabsorbent material in combination with the fibrous matrix in a dosage level of between about 40 and about 60 percent, the absorbent composite having a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals, substantially equal to or less than the first composite-bed friction angle. The first composite-bed friction angle may be about 27 degrees or less.

The absorbent composite of the present invention may comprise a fibrous matrix and a water swellable, water insoluble superabsorbent material in combination with the fibrous matrix in a dosage level of between about 60 and about 80 percent, the absorbent composite having a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals, substantially equal to or less than the first composite-bed friction angle. The first composite-bed friction angle may be about 25 degrees or less.

The absorbent composite of the present invention may comprise a fibrous matrix and a water swellable, water insoluble superabsorbent material in combination with the fibrous matrix in a dosage level of between about 20 and about 80 percent, the absorbent composite having a composite-bed cohesion value when swollen in a NaCl solution having a NaCl concentration of about 20% by weight for one hour under an external load of 2,000 Pascals of about 1,200 Pascals or less.

The absorbent composite of the present invention may comprise a fibrous matrix and a water swellable, water insoluble superabsorbent material in combination with the fibrous matrix in a dosage level of between about 20 and about 40 percent, the absorbent composite having a first composite-bed friction angle when swollen in a NaCl solution having a NaCl concentration of about 5% by weight for one hour under an external load of 2,000 Pascals and composite-bed friction angles, when swollen in a NaCl solution having a NaCl concentration of less than about 5% by weight for one hour under an external load of 2,000 Pascals, substantially equal to or greater than the first composite-bed friction angle. Then first composite-bed friction angle may be about 39 degrees or greater.

The absorbent composite of the present invention may comprise a fibrous matrix and a water swellable, water insoluble superabsorbent material in combination with the fibrous matrix in a dosage level of between about 40 and about 60 percent, the absorbent composite having a first composite-bed friction angle when swollen in a NaCl solution having a NaCl concentration of about 5% by weight for one hour under an external load of 2,000 Pascals and composite-bed friction angles, when swollen in a NaCl solution having a NaCl concentration of less than about 5% by weight for one hour under an external load of 2,000 Pascals, substantially equal to or greater than the first composite-bed friction angle. The first composite-bed friction angle may be about 35 degrees or greater.

The absorbent composite of the present invention may comprise a fibrous matrix and a water swellable, water insoluble superabsorbent material in combination with the fibrous matrix in a dosage level of between about 60 and about 80 percent, the absorbent composite having a first composite-bed friction angle when swollen in a NaCl solution having a NaCl concentration of about 5% by weight for one hour under an external load of 2,000 Pascals and composite-bed friction angles, when swollen in a NaCl solution having a NaCl concentration of less than about 5% by weight for one hour under an external load of 2,000 Pascals, substantially equal to or greater than the first composite-bed friction angle. The first composite-bed friction angle may be about 33 degrees or greater.

The absorbent composite of the present invention may comprise a fibrous matrix and a water swellable, water insoluble superabsorbent material in combination with the fibrous matrix in a dosage level of between about 20 and about 80 percent, the absorbent composite having a composite-bed cohesion value when swollen in a NaCl solution having a NaCl concentration of about 20% by weight for one hour under an external load of 2,000 Pascals of about 4,500 Pascals or greater.

The absorbent composite of the present invention may comprise a fibrous matrix and a water swellable, water insoluble superabsorbent material in combination with the fibrous matrix in a dosage level of between about 20 and about 80 percent, the absorbent composite having a composite-bed cohesion value when swollen in a NaCl solution having a NaCl concentration of about 5% by weight for one hour under an external load of 2,000 Pascals of about 3,000 Pascals or greater.

The absorbent composite of the present invention may comprise a fibrous matrix and a water swellable, water insoluble superabsorbent material in combination with the fibrous matrix in a dosage level of between about 20 and about 40 percent, the absorbent composite having a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals, substantially equal to or greater than the first composite-bed friction angle. The first composite-bed friction angle may be about 30 degrees or less.

The absorbent composite of the present invention may comprise a fibrous matrix and a water swellable, water insoluble superabsorbent material in combination with the fibrous matrix in a dosage level of between about 40 and about 60 percent, the absorbent composite having a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals, substantially equal to or greater than the first composite-bed friction angle. The first composite-bed friction angle may be about 27 degrees or less.

The absorbent composite of the present invention may comprise a fibrous matrix and a water swellable, water insoluble superabsorbent material in combination with the fibrous matrix in a dosage level of between about 60 and about 80 percent, the absorbent composite having a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals, substantially equal to or greater than the first composite-bed friction angle. The first composite-bed friction angle may be about 25 degrees or less.

These and other features, aspects, and advantages of the present invention will become better understood with regard to the following description, appended claims, and accompanying drawings.

### BRIEF DESCRIPTION OF DRAWINGS OF EXAMPLES AND/OR REPRESENTATIVE EMBODIMENTS

FIG. 1 shows an example of a response of a porous medium to a stress (i.e., a force per unit area) acting on the medium.

FIG. 2 shows an example of the state of stress of an arbitrary element at equilibrium in a porous medium.

FIG. 3 shows an example of an arbitrary element and the normal forces and shear forces acting on a plane passing through the arbitrary element.

FIG. 4 shows an example of a Mohr Circle on a plot of shear stress (y axis) versus normal stress (x axis).

FIG. 5 shows an example of a sequence of Mohr Circles corresponding to one possible stress path on a plot of shear stress (y axis) versus normal stress (x axis).

FIG. 6 shows an example of Mohr Circles in relation to a Mohr-Coulomb failure envelope on a plot of shear stress (y axis) versus normal stress (x axis).

FIG. 7 shows another example of Mohr Circles in relation to a Mohr-Coulomb failure envelope on a plot of shear stress (y axis) versus normal stress (x axis).

FIG. 8 shows an example of a friction-angle measuring device, in this case a Jenike-Schulze ring-shear tester, available in the U.S. from Jenike-Johanson, a business having offices in Westford, Mass.

### DEFINITIONS

Within the context of this specification, each term or phrase below will include the following meaning or meanings.

“Absorbency Under Load” (AUL) refers to the measure of the liquid retention capacity of a material under mechanical load. It is determined by a test which measures the amount, in grams, of a 0.9% by weight aqueous sodium chloride solution a gram of material may absorb in 1 hour under an applied load or restraining pressure of about 0.3 pound per square inch (2,000 Pascals). A procedure for determining AUL is provided in U.S. Pat. No. 5,601,542, which is incorporated by reference in its entirety in a manner consistent herewith.

“Absorbent article” includes, without limitation, diapers, training pants, swim wear, absorbent underpants, baby wipes, incontinence products, feminine hygiene products and medical absorbent products (for example, absorbent medical garments, underpads, bandages, drapes, and medical wipes).

“Fiber” and “Fibrous Matrix” includes, but is not limited to natural fibers, synthetic fibers and combinations thereof. Examples of natural fibers include cellulosic fibers (e.g., wood pulp fibers), cotton fibers, wool fibers, silk fibers and the like, as well as combinations thereof. Synthetic fibers can include rayon fibers, glass fibers, polyolefin fibers, polyester fibers, polyamide fibers, polypropylene. As used herein, it is understood that the term “fibrous matrix” includes a plurality of fibers.

“Free Swell Capacity” refers to the result of a test which measures the amount in grams of an aqueous 0.9% by weight sodium chloride solution that a gram of material may absorb in 1 hour under negligible applied load.

“Fiber-bed friction angle” refers to the friction angle of a fiber or fiber material in a fiber bed as measured with a Jenike-Shulze ring shear tester or other friction angle measuring technique. Unless otherwise specified, the determination is done with wetted fiber. For purposes of this application, the fiber is considered to be wetted when the fiber is brought to a saturation level, with 0.9% sodium chloride solution (sodium chloride dissolved in distilled water), which corresponds to about 0.2 grams or more of 0.9% sodium chloride solution per gram of oven-dry fiber. The oven-dry weight of fiber is determined by placing a small quantity of fiber in an oven at 105 degrees Celsius for 2-4 hours. The dried fiber is placed in a dessicator with a desiccant until it is cool. The fiber is then weighed. For purposes of this application, the fiber is considered to be dry when the fiber is below 0.2 grams of moisture per grams of dry fibers.

“Gel-bed friction angle” refers to the friction angle of a superabsorbent material in a gel-bed as measured with a Jenike-Shulze ring shear tester or other friction angle measuring technique.

“Composite-bed friction angle” refers to the friction angle of a composite material as measured with a Jenike-Shulze ring shear tester or other friction angle measuring technique (see procedure herein).

For purposes of this application, “fiber-bed cohesion,” “fiber-bed effective cohesion,” and “fiber-bed cohesion value” refers to cohesion of a fiber or fiber material in a fiber bed as measured with a Jenike-Shulze ring shear tester or other measuring technique. Unless otherwise specified, the determination is done with wetted fiber. For purposes of this application, the fiber is considered to be wetted when the fiber is brought to a saturation level, with 0.9% sodium chloride solution (sodium chloride dissolved in distilled water), which corresponds to about 0.5 grams or more of 0.9% sodium chloride solution per gram of oven-dry fiber. The oven-dry weight of fiber is determined by placing a small quantity of fiber in an oven at 105 degrees Celsius for 2-4 hours. The dried fiber is placed in a dessicator with a dessicant until it is cool. The fiber is then weighed.

“Gel-bed Cohesion,” “gel-bed effective cohesion,” and “gel-bed cohesion value” refers to cohesion of a superabsorbent material in a gel-bed as measured with a Jenike-Shulze ring shear tester or other similar measuring technique.

“Composite-bed cohesion,” “composite-bed effective cohesion,” and “composite-bed cohesion value” refers to cohesion of a composite material in a composite bed as measured with a Jenike-Shulze ring shear tester or other similar measuring technique.

“Gradient” refers to a graded change in the magnitude of a physical quantity, such as the quantity of superabsorbent material present in various locations of an absorbent pad, or other pad characteristics such as mass, density, or the like.

“Fiber bed” or “fiber-bed” refers to an amount of fiber within a container such as a ring shear cell.

“Gel bed” or “gel-bed” refers to an amount of superabsorbent material within a container such as a ring shear cell.

“Composite bed” or “composite-bed” refers to an amount of superabsorbent material and fiber within a container such as a ring shear cell.

“High yield pulp fibers” are those papermaking fibers produced by pulping processes providing a yield of about 65 percent or greater, more specifically about 75 percent or greater, and still more specifically from about 75 to about 95 percent. Such pulping processes include bleached chemithermomechanical pulp (BCTMP), chemithermomechanical pulp (CTMP), pressure/pressure thermomechanical pulp (PTMP), thermomechanical pulp (TMP), thermomechanical chemical pulp (TMCP), high yield sulphite pulps, and high yield kraft pulps, all of which leave the resulting fibers with high levels of lignin. Suitable high-yield pulp fibers are characterized by being comprised of comparatively whole, relatively undamaged tracheids, high freeness (over 250 CSF), and low fines content (less than 25 percent by the Britt jar test).

“Homogeneously mixed” refers to the uniform mixing of two or more substances within a composition, such that the magnitude of a physical quantity of each of the substances remains substantially consistent throughout the composition.

“Incontinence products” includes, without limitation, absorbent underwear for children, absorbent garments for children or young adults with special needs such as autistic children or others with bladder/bowel control problems as a result of physical disabilities, as well as absorbent garments for incontinent older adults.

“Meltblown fiber” means fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers which may be continuous or discontinuous, are generally smaller than about 0.6 denier, and are generally self bonding when deposited onto a collecting surface. Meltblown fibers used in the present invention are suitably substantially continuous in length.

“Mohr circle” refers to a graphical representation of the state of stress within a material subjected to one or more forces. Mohr circles are described in more detail below.

“Mohr failure envelope” refers to the failure shear stress at the failure plane as a function of the normal stress on that failure or shear plane. Mohr failure envelopes are described in more detail below.

“Polymers” include, but are not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and atactic symmetries. “Superabsorbent” or “superabsorbent material” refers to a water-swellable, water-insoluble organic or inorganic material capable, under the most favorable conditions, of absorbing at least about 10 times its weight and, more particularly, at least about 20 times its weight in an aqueous solution containing 0.9 weight percent sodium chloride. The superabsorbent materials may be natural, synthetic and modified natural polymers and materials. In addition, the superabsorbent materials may be inorganic materials, such as silica gels, or organic compounds such as cross-linked polymers. The superabsorbent materials of the present invention may embody various structure configurations including particles, fibers, flakes, and spheres.

“Spunbonded fiber” refers to small diameter fibers which are formed by extruding molten thermoplastic material as filaments from a plurality of fine capillaries of a spinnerette having a circular or other configuration, with the diameter of the extruded filaments then being rapidly reduced as by, for example, in U.S. Pat. No. 4,340,563 to Appel et al.; U.S. Pat. No. 3,692,618 to Dorschner et al.; U.S. Pat. No. 3,802,817 to Matsuki et al.; U.S. Pat. Nos. 3,338,992 and 3,341,394 to Kinney; U.S. Pat. No. 3,502,763 to Hartmann; U.S. Pat. No. 3,502,538 to Petersen; and, U.S. Pat. No. 3,542,615 to Dobo et al., each of which is incorporated by reference in its entirety in a manner consistent herewith. Spunbond fibers are quenched and generally not tacky when they are deposited onto a collecting surface. Spunbond fibers are generally continuous and often have average deniers larger than about 0.3, more particularly, between about 0.6 and 10.

These terms may be defined with additional language in the remaining portions of the specification.

### Overview of Continuum Mechanics, Mohr Circles, and Mohr-Coulomb Failure Theory

Given that our discovery is described using tools and terminology from mechanics, an overview of continuum mechanics, Mohr circles, and Mohr-Coulomb failure theory is provided for convenience. It should be understood that this overview is for purposes of explanation only—it provides an analytic framework for characterizing the present invention, and should not be viewed as limiting the present invention disclosed herein.

Absorbent articles and composites are porous by nature. The open space between the various ingredients that make up the composite (e.g., superabsorbent material and fibers) is commonly referred to as void space or pore space. Pore space acts to store liquids and/or provide a conduit or pathway for transporting liquid throughout the absorbent composite or article. The volume of pore space per unit volume of absorbent composite is commonly referred to as “porosity.” Generally absorbency performance is improved by increasing porosity. For example, permeability of an absorbent composite—i.e., the ability of the composite to facilitate liquid transport—increases with increasing porosity (other factors, such as specific surface area and tortuosity, being equal).

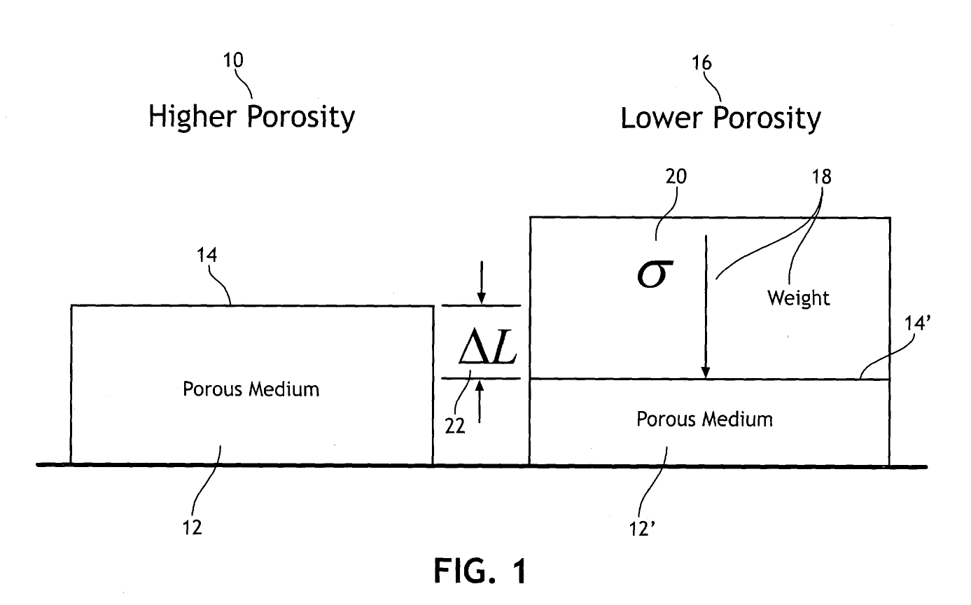

The application of a stress to a porous medium, such as an absorbent composite or article, is known to cause a volumetric deformation of the medium as a whole, as well as shear deformation in the case of anisotropic stresses. FIG. 1 depicts an example of a volumetric deformation of a porous medium. The left-most image of FIG. 1 is labeled “Higher Porosity” 10 and shows a porous medium 12 without a weight applied to the uppermost planar surface 14 of the porous medium 12 (with the uppermost planar area having some discrete area). The right-most image of FIG. 1 is labeled “Lower Porosity” 16 and shows the same porous medium 12′ with a weight 18 applied to the uppermost planar surface 14′ of the porous medium 12′. In response to the placement of the weight 18, which produces a stress, or normal force per unit area, σ 20, the thickness decreases (as denoted by Δ L 22). (Note: for purposes of the present invention, compressive stresses are represented as having positive values.)

For a porous medium 12 made up of individual ingredients such as superabsorbent particles and fibers (e.g., an absorbent composite), the thickness change of the porous medium 12 as a whole, Δ L 22, likely does not result from a reduction in the individual dimensions of individual particles and fibers (reductions in these individual thicknesses would likely be small or negligible). Instead, the decrease in the thickness of the porous medium 12 as a whole, Δ L 22, results from a reduction in porosity (or, analogously, void volume). Accordingly, in the example depicted in FIG. 1, an increase in stress, or normal force per unit area, σ 20, reduces the thickness Δ L 22 of the porous medium 12 as a whole, and reduces the porosity of the porous medium 12. (Note: If, in FIG. 1, a fluid in the pores is a compressible gas, then a normal stress acting on the surface of the porous medium 12 would: compress the gas within the pores; or cause a portion of the gas within the pores to exit the porous medium 12; or, some combination thereof. If, in this same FIG. 1, a fluid in the pores is an incompressible liquid, then a normal stress acting on the surface of the porous medium 12 would cause a portion of the liquid to exit the porous medium 12.)

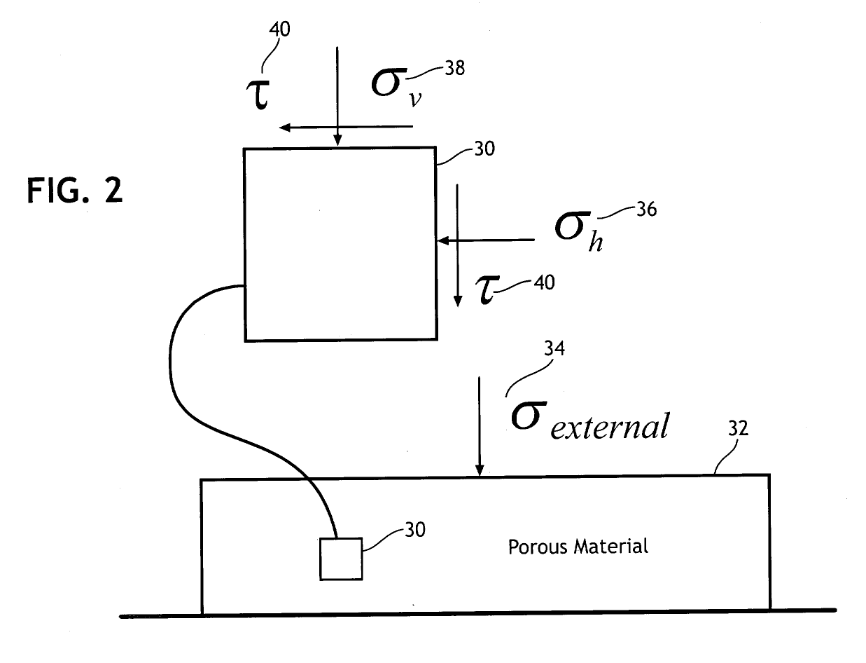

The porous medium 12 of FIG. 1 may be examined further to analyze the stresses acting on an arbitrary element 30 within the porous medium 12. FIG. 2 illustrates the state of stress of an arbitrary element 30—here represented by the face of a cube—at equilibrium (the arbitrary element is within a porous medium 32 being subjected to an external stress σₑₓₜₑᵣₙₐₗ 34). For present purposes, the arbitrary element 30 within the porous medium 32 is treated as a continuum. In FIG. 2, the state of stress is represented by two normal components of stress, σₕ 36 acting horizontally on a face of the cube and σᵥ 38 acting vertically on another face of the cube, as well as a shear stress τ40. The normal components of stress 36 are perpendicular to the faces of the arbitrary element 30, whereas the shear stresses 40 are parallel to the faces of the arbitrary element 30.

It should be noted that if the shear stresses 40 are zero (i.e., τ=0), then the two normal stresses 36 are referred to as principal stresses. Furthermore, when τ=0, then the larger of the two normal stresses 36 is called the major principal stress while the other is called the minor principal stress. For the present discussion, the two stresses are assumed to be principal stresses, with σₕ≧σᵥ.

There are generally at least two contributions to stress generation that combine to produce principal stresses such as those identified in FIG. 2. The first is an external stress 34, possibly non-uniform, acting on the boundary of the porous medium 32. This stress is transmitted throughout the porous medium 32 in accordance with well known force-balance equations. The second contribution arises due to swelling of components that make up the porous medium 32 (e.g., a superabsorbent material). For example, the swelling of blocks, or elements, immediately adjacent to the arbitrary element 30 depicted in FIG. 2, may cause an “internally” generated stress acting on or along the arbitrary element 30 as other elements attempt to expand against it and each other.

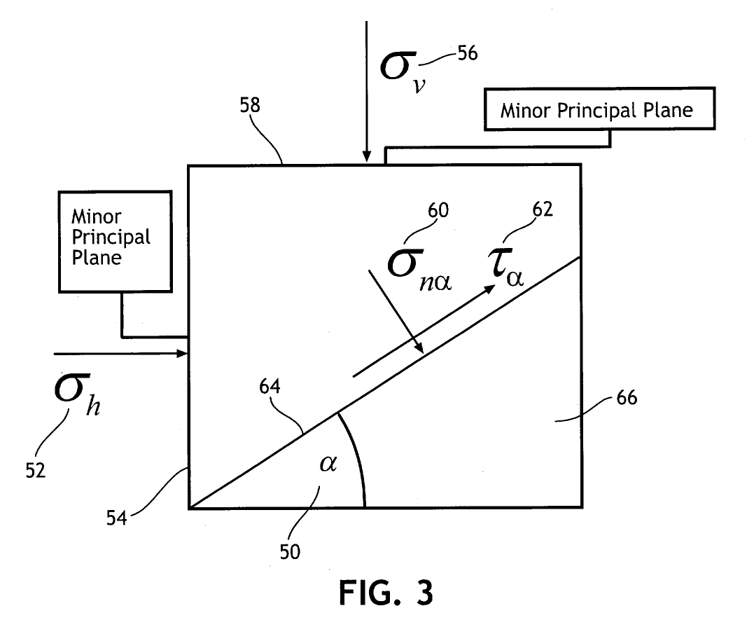

As stated above, when the stresses acting on an arbitrary element 30, such as that depicted in FIG. 2, are principal stresses, there are no shear stresses 40 acting on the faces of the arbitrary element 30. There is, however, shear stress 40 acting on other imaginary planes passing through the depicted arbitrary element 30—planes oriented at some angle α 50 away from horizontal, 0<α<90°, as shown in FIG. 3. FIG. 3 depicts a major principal stress σₕ 52 acting on a major principal plane 54, and a minor principal stress σᵥ 56 acting on a minor principal plane 58. A normal stress σ_{nα} 60 and a shear stress τ_{α} 62 act on the imaginary or arbitrary plane 64 oriented at angle α 50 away from horizontal.

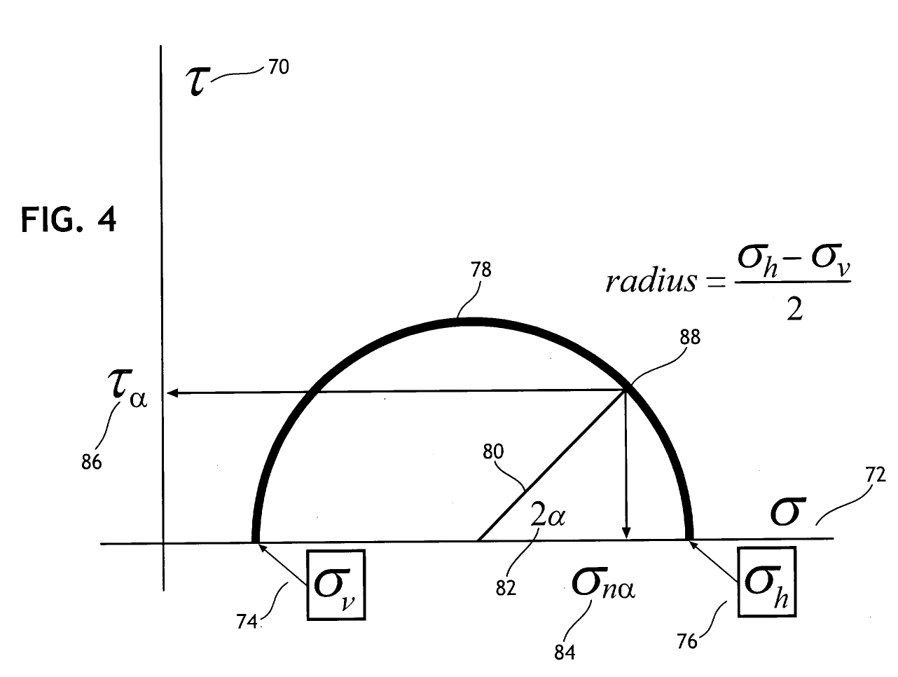

Obtaining the shear and normal forces 62 and 60, respectively, acting on the arbitrary plane 64 passing through the element 66 depicted in FIG. 3 is simplified by using the graphical approach of the Mohr circle, as illustrated in FIG. 4. FIG. 4 shows a plot of shear stress (y-axis) 70 as a function of normal stress (x-axis) 72. For purposes of the present discussion the principal stresses are assumed to be known (e.g., by calculation or measurement). The x-y coordinates of the minor principal stress σᵥ 74 and the major principal stress τ 76 lie on the x-axis (i.e., where the shear stress τ 70 is equal to zero). A semi-circle 78 is drawn such that the coordinates of the minor and major principal stresses 74 and 76, respectively, correspond to the end points of the arc defining the perimeter of the semi-circle 78. The radius of this semi-circle 78 equals one-half of the difference between the major principal stress σₕ 76 and the minor principal stress σᵥ 74. By constructing a radial line segment 80 at an angle 2 α 82 from the x-axis, with one end of the radial line segment 80 corresponding to the center of the semi-circle 78, and other end corresponding to a point on the semi-circle arc closest to the major principal stress, both the normal stress, σ_{α} 84, and the shear stress τ_{α} 86 are obtained at the intersection 88 of the radial line segment 80 with the Mohr semi-circle 78.

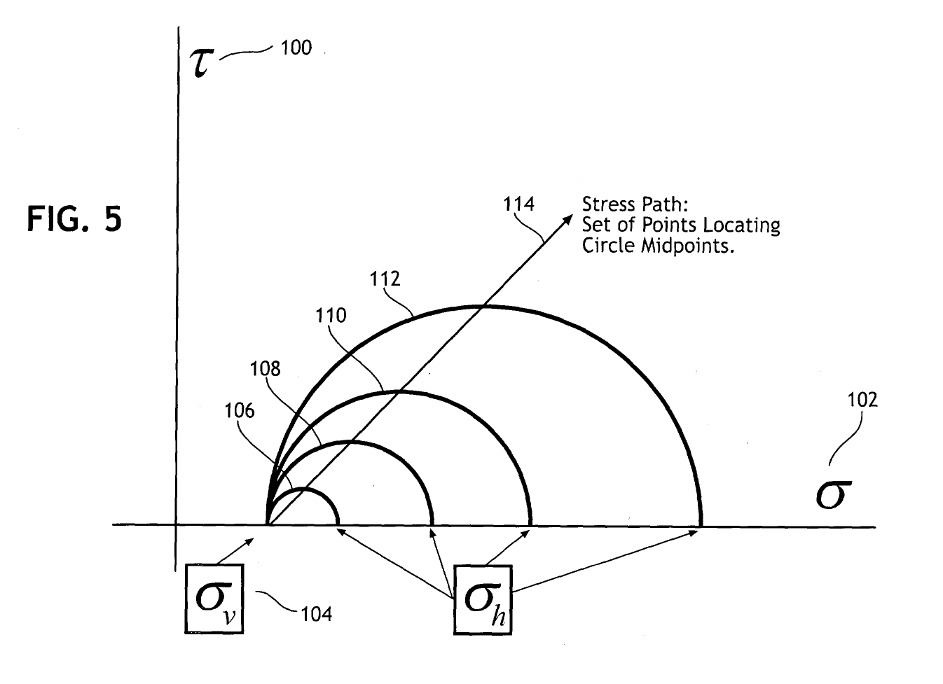

FIG. 5 depicts one example of stress evolution for a porous medium that employs one or more swelling components (e.g., a particulate superabsorbent material). The y-axis again corresponds to shear stress τ 100, and the x-axis again corresponds to normal stress σ 102. If the minor principal stress σᵥ 104 acting on an arbitrary element from the porous medium remains unchanged, then stress development (which would accompany, for example, swelling of superabsorbent material) may be viewed as a family of Mohr circles 106, 108, 110, and 112, all of which have the same minor principal stress σᵥ 104. The progression of Mohr circles 106, 108, 110, and 112 is commonly referred to as a stress path 114—more precisely, the line passing through the set of Mohr circles 106, 108, 110, and 112 at points simultaneously locating the maximum shear stress and mean stress for each Mohr circle 106, 108, 110, and 112.

The center of each Mohr circle 106, 108, 110, and 112, which equates to the mean stress, determines the volumetric deformation of pore space contained within a particular arbitrary element, and may correspond to the approximate stress experienced by superabsorbent materials.

Stresses in a porous medium are not likely to increase indefinitely—rather, failure will take place, accompanied by sliding along particular failure planes (e.g., at the interface between superabsorbent material and fiber; or at the interface between individual particles of superabsorbent material; etc.). The Mohr-Coulomb failure criterion states that a shear force acting on a plane at failure will be linearly proportional to the normal force acting on that same plane, again at failure. Hence, Mohr-Coulomb theory provides a failure limit, or envelope, beyond which stable states of stress do not exist. If a line corresponding to this failure limit is superimposed on a plot of shear stress and normal stress depicting a Mohr circle 106, 108, 110, and 112 (which may be thought of as corresponding to a given state or degree of swelling for a porous medium employing a superabsorbent material), then the Mohr circle 106, 108, 110, and 112 may only increase in radius (e.g., by additional swelling of the porous medium and/or superabsorbent material employed by the porous medium) to the extent that it becomes tangent to this linear envelope. It should be noted that the failure envelope may be determined empirically using a tester, such as the Jenike-Schulz ring-shear tester, by determining the shear stress at failure for a given normal stress acting on a bed of material (e.g., a fiber bed; or a gel bed of superabsorbent material). By plotting a number of shear stresses at failure for a number of different normal stresses, the Mohr-Coulomb failure envelope (or line or limit) may be determined.

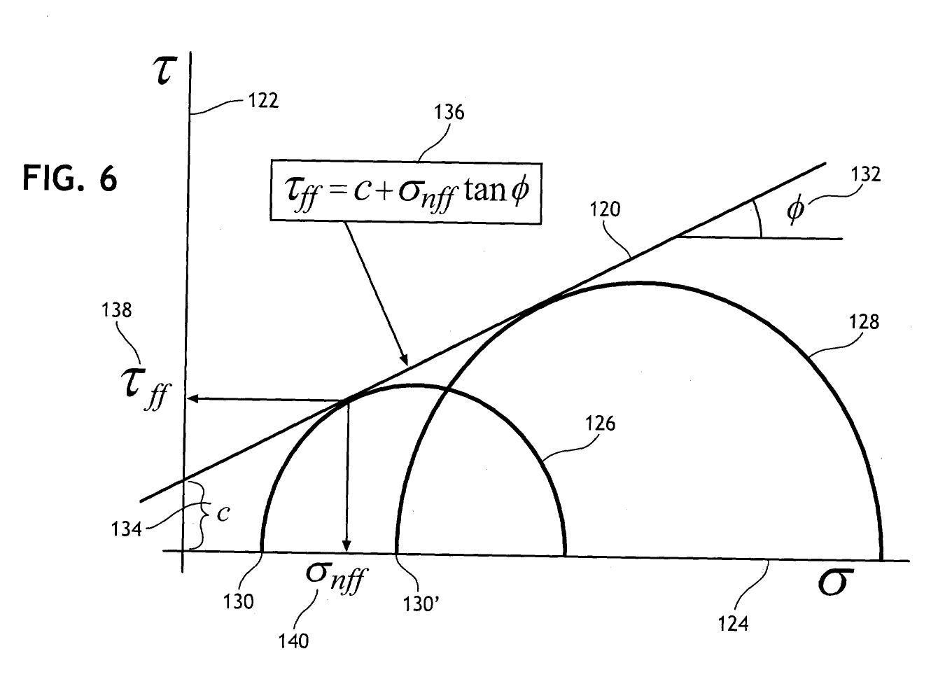

FIG. 6 depicts a linear failure envelope 120 on a plot of shear stress τ 122 versus normal stress σ 124. On this plot are depicted two Mohr circles 126 and 128, with each Mohr circle 126 and 128 having a different value of initial stress—that is, two different values of the minor principal stress σᵥ 130 and 130′. The friction angle φ 132 and cohesion c 134 are properties of a particular material (e.g., an absorbent composite—i.e., a composite bed-comprising fiber and superabsorbent material; a gel bed of swollen, particulate superabsorbent material; etc.). The tangent of the friction angle φ 132, which is equivalent to the coefficient of static friction from elementary physics, measures the extent to which an increasing normal force permits a larger maximum shear stress. Cohesion c 134 represents the amount of shear stress a material will tolerate before failure in the absence of any normal force on the proposed failure plane. An increase in any one of the three parameters—friction angle φ 132, cohesion c 134, or minor principle stress σᵥ 130 and 130′—will permit the development of larger stresses in a porous material—i.e., a larger Mohr circle. Friction angle φ 132 and cohesion c 134 are material dependent and may be measured (e.g., using the test and methodology disclosed herein). FIG. 6 also depicts the mathematical relationship τ_{ff}=c+σ_{nff}(tan φ) 136, which relates friction angle φ 132, cohesion c 134, shear stress at failure τ_{ff} 138, and normal stress at failure σ_{nff} 140. (Note: for purposes of this disclosure, σ_{nff} is equivalent to σ_{ff}, with both terms referring to a normal stress acting on a failure plane at failure.) This relationship is described in more detail below in the Detailed Description section.

As stated earlier, it is generally advantageous to minimize or decrease the reduction of porosity, or void volume, that results from the application of a compressive stress to an absorbent article. By choosing materials that limit stress increases (e.g., fiber having controlled fiber-bed friction angle or fiber-bed cohesion values such that one or both of these properties is relatively low; superabsorbent having controlled gel-bed friction angle or gel-bed cohesion values such that one or both of these properties is relatively low; or both) the magnitude of porosity reductions may be decreased. For example, low, controlled fiber-bed friction angle fiber and/or low, controlled gel-bed friction angle superabsorbent material will promote the onset of failure before stresses rise to values that cause significant losses of porosity, and therefore permeability. An additional benefit of providing stress relief through these low friction-angle ingredients is that any superabsorbent materials employed with fiber in a composite will retain a larger portion of its free-swell capacity—since it is well known that superabsorbent capacity decreases with increasing loading. It should be noted, however, that in some contexts—e.g., an absorbent composite having a high porosity—it may be advantageous to employ a fiber having a high, controlled fiber-bed friction angle and/or fiber-bed cohesion value; a superabsorbent having a high, controlled gel-bed friction angle and/or gel-bed cohesion value, or both; or some combination of these ingredients; thereby “locking in” the high porosity.

Additional detail regarding continuum mechanics, Mohr circles, and Mohr-Coulomb failure theory may be found in a number of textbooks and other publications, including, for example, ROBERT D. HOLTZ AND WILLIAM D KOVACS, AN INTRODUCTION TO GEOTECHNICAL ENGINEERING 431-84 (Prentice Hall, Inc. 1981).

### DETAILED DESCRIPTION OF REPRESENTATIVE EMBODIMENTS

The present invention encompasses composites comprising fiber and superabsorbent materials having controlled composite-bed friction angles and/or controlled composite-bed cohesion values. Such composites will generally comprise: a superabsorbent having a desired gel-bed friction angle and/or gel-bed cohesion; a fiber having a desired fiber-bed friction angle and/or fiber-bed cohesion; or some combination thereof. Composites of the present invention may be made with any of the forming methods typically used to prepare absorbent or fibrous composites, such as air laying, air forming, wet forming, and the like.

Composites of the present invention may contain superabsorbent material, in relatively high quantities in some cases, in various forms such as superabsorbent fibers and/or superabsorbent particles, homogeneously mixed with a matrix material, such as cellulose fluff pulp. The mixture of superabsorbent material and cellulose fluff pulp may be homogeneous or non-homogeneous throughout the absorbent composite. The superabsorbent material may be strategically located within the absorbent composite, such as forming a gradient within the fiber matrix. For example, more superabsorbent material may be present at one end of the absorbent composite than at an opposite end of the absorbent composite. Alternatively, more superabsorbent material may be present along a top surface of the absorbent composite than along a bottom surface of the absorbent composite or more superabsorbent material may be present along the bottom surface of the absorbent composite than along the top surface of the absorbent composite. One skilled in the art will appreciate the various embodiments available for absorbent composites.

Absorbent composites of the present invention comprising a superabsorbent material typically include a matrix which contains the superabsorbent material. The matrix is often made from a fibrous material or foam material, but one skilled in the art will appreciate the various embodiments of the composite matrix. One such fibrous matrix is made of a cellulose fluff pulp. The cellulose fluff pulp suitably includes wood pulp fluff. The cellulose pulp fluff may be exchanged, in whole or in part, with synthetic, polymeric fibers (e.g., meltblown fibers). Synthetic fibers are not required in the absorbent composites of the present invention, but may be included. One preferred type of wood pulp fluff is identified with the trade designation CR1654, available from Bowater, Childersburg, Ala., U.S.A., and is a bleached, highly absorbent wood pulp containing primarily soft wood fibers. The cellulose fluff pulp may be homogeneously or non-homogeneously mixed with the superabsorbent material. Within the absorbent article, the mixed fluff and superabsorbent material may be selectively placed into desired zones of higher concentration to better contain and absorb body exudates. For example, the mass of the mixed fluff and superabsorbent materials may be controllably positioned such that more basis weight is present in a front portion of the pad than in a back portion of the pad.

Absorbent composites of the present invention may suitably contain between about 5 to about 95 mass % of superabsorbent material, based on the total weight of the fiber, the superabsorbent material, and/or any other component. Optionally, the mass composition of the superabsorbent material in the absorbent composite may be from about 20 to about 80%. Additionally, the mass composition of the superabsorbent material in the absorbent composite may be from about 40 to about 60%.

Suitable superabsorbent materials that may be employed with in composites of the present invention may be selected from natural, synthetic, and modified natural polymers and materials. The superabsorbent materials may be inorganic materials, such as silica gels, or organic compounds, including natural materials such as agar, pectin, guar gum, and the like, as well as synthetic materials, such as synthetic hydrogel polymers. Such hydrogel polymers include, for example, alkali metal salts of polyacrylic acids; polyacrylamides; polyvinyl alcohol; ethylene maleic anhydride copolymers; polyvinyl ethers; hydroxypropylcellulose; polyvinyl morpholinone; polymers and copolymers of vinyl sulfonic acid, polyacrylates, polyacrylamides, polyvinyl pyridine; polyamines; and, combinations thereof. Other suitable polymers include hydrolyzed acrylonitrile grafted starch, acrylic acid grafted starch, and isobutylene maleic anhydride copolymers and combinations thereof. The hydrogel polymers are suitably lightly crosslinked to render the material substantially water-insoluble. Crosslinking may, for example, be by irradiation or by covalent, ionic, Van der Waals, or hydrogen bonding. The superabsorbent materials may be in any form suitable for use in absorbent structures, including, particles, fibers, flakes, spheres, and the like.

Typically, a superabsorbent polymer is capable of absorbing at least about 10 times its weight in a 0.9 weight percent aqueous sodium chloride solution, and particularly is capable of absorbing more than about 20 times its weight in 0.9 weight percent aqueous sodium chloride solution. Superabsorbent polymers are available from various commercial vendors, such as Dow Chemical Company located in Midland, Mich., U.S.A., and Stockhausen Inc., Greensboro, N.C., USA. Other superabsorbent polymers are described in U.S. Pat. No. 5,601,542 issued Feb. 11, 1997, to Melius et al.; U.S. patent application Ser. No. 09/475,829 filed in December 1999 and assigned to Kimberly-Clark Corporation; and, U.S. patent application Ser. No. 09/475,830 filed in December 1999 and assigned to Kimberly-Clark Corporation, each of which is hereby incorporated by reference in a manner consistent herewith.

Other examples of commercial superabsorbent materials polyacrylate materials available from Stockhausen under the tradename FAVOR®. Examples include FAVOR® SXM 77, FAVOR® SXM 880, and FAVOR® SXM 9543. Other polyacrylate superabsorbent materials are available from Dow Chemical, USA under the tradename DRYTECH®, such as DRYTECH® 2035.

Superabsorbent materials may be in the form of particles which, in the unswollen state, have maximum cross-sectional diameters typically within the range of from about 50 microns to about 1,000 microns, suitably within the range of from about 100 microns to about 800 microns, as determined by sieve analysis according to American Society for Testing Materials (ASTM) Test Method D-1921. It is understood that the particles of superabsorbent material, falling within the ranges described above, may include solid particles, porous particles, or may be agglomerated particles including many smaller particles agglomerated into particles within the described size ranges.

Fibers suitable for use in the present invention are known to those skilled in the art. Examples of fibers suitable for use in the present invention include, cellulosic fibers such as wood pulp, cotton linters, cotton fibers and the like; synthetic polymeric fibers such as polyolefin fibers, polyamide fibers, polyester fibers, polyvinyl alcohol fibers, polyvinyl acetate fibers, synthetic polyolefin wood pulp fibers, and the like; as well as regenerated cellulose fibers such as rayon and cellulose acetate microfibers. Mixtures of various fiber types are also suitable for use. For example, a mixture of cellulosic fibers and synthetic polymeric fibers may be used. As a general rule, the fibers will have a length-to-diameter ratio of at least about 2:1, suitably of at least about 5:1. As used herein, “diameter” refers to a true diameter if generally circular fibers are used or to a maximum transverse cross-sectional dimension if non-circular, e.g., ribbon-like, fibers are used. The fibers will generally have a length of from about 0.5 millimeter to about 25 millimeters, suitably from about 1 millimeter to about 6 millimeters. Fiber diameters will generally be from about 0.001 millimeter to about 1.0 millimeter, suitably from about 0.005 millimeter to about 0.05 millimeter. For reasons such as economy, availability, physical properties, and ease of handling, cellulosic wood pulp fibers are suitable for use in the present invention.

Other fibers useful for purposes of the present invention are resilient fibers that include high-yield pulp fibers (further discussed below), flax, milkweed, abaca, hemp, cotton, or any of the like that are naturally resilient or any wood pulp fibers that are chemically or physically modified, e.g. crosslinked or curled, that have the capability to recover after deformation from preparing the composite, as opposed to non-resilient fibers which remain deformed and do not recover after preparing the composite.

Absorbent composites may also contain any of a variety of chemical additives or treatments, fillers or other additives, such as clay, zeolites and/or other odor-absorbing material, for example activated carbon carrier particles or active particles such as zeolites and activated carbon. Absorbent composites may also include binding agents, such as crosslinkable binding agents or adhesives, and/or binder fibers, such as bicomponent fibers. Absorbent composites may or may not be wrapped or encompassed by a suitable tissue wrap that maintains the integrity and/or shape of the absorbent composite.

The structure and components of absorbent composites are designed to take up fluids and absorb them. The porosity of the fiber matrix allows fluid to penetrate the absorbent composite. When the absorbent composite includes superabsorbent material, the fiber matrix facilitates penetration of fluid into the composite and in contact with superabsorbent material, which absorbs the fluids. The superabsorbent material swells as the superabsorbent material absorbs fluids. The swelling of the superabsorbent material may be influenced by several factors such as the surrounding matrix material and pressures (i.e., a force per unit area, or stress) from the absorbent article user. The surrounding matrix fibers and/or superabsorbent materials and the pressures on the superabsorbent material may inhibit the swelling of the superabsorbent material, thus stopping absorbency, and thereby the absorbent composite, from reaching full free swell capacity. Also, as described above, stresses acting on an absorbent composite, such as an absorbent composite employing a superabsorbent material, may reduce porosity and/or permeability of the absorbent composite.

To the extent possible during swelling, superabsorbent materials may move within the composite matrix to positions that allow the superabsorbent to obtain greater swelling. Superabsorbent materials may rotate and/or translate so as to fit within voids in the composite matrix which allows the absorbent particle to swell readily against surrounding matrix and reach greater swelling potentials. Moreover, additional voids/void space may be created by overall expansion of the absorbent composite. Upon moving within the fiber matrix, the superabsorbent materials will contact and rub against other components of the absorbent composite, including matrix fibers and/or other superabsorbent materials. The surface mechanics of the superabsorbent material and the surrounding matrix components may determine the amount of superabsorbent material structure rotation and/or translation and thus may affect: (1) the swelling capacity of the superabsorbent material, and therefore the absorbent composite; and, (2) the level of stress buildup in an absorbent composite employing the superabsorbent, which in turn affects the porosity and permeability of the absorbent composite.

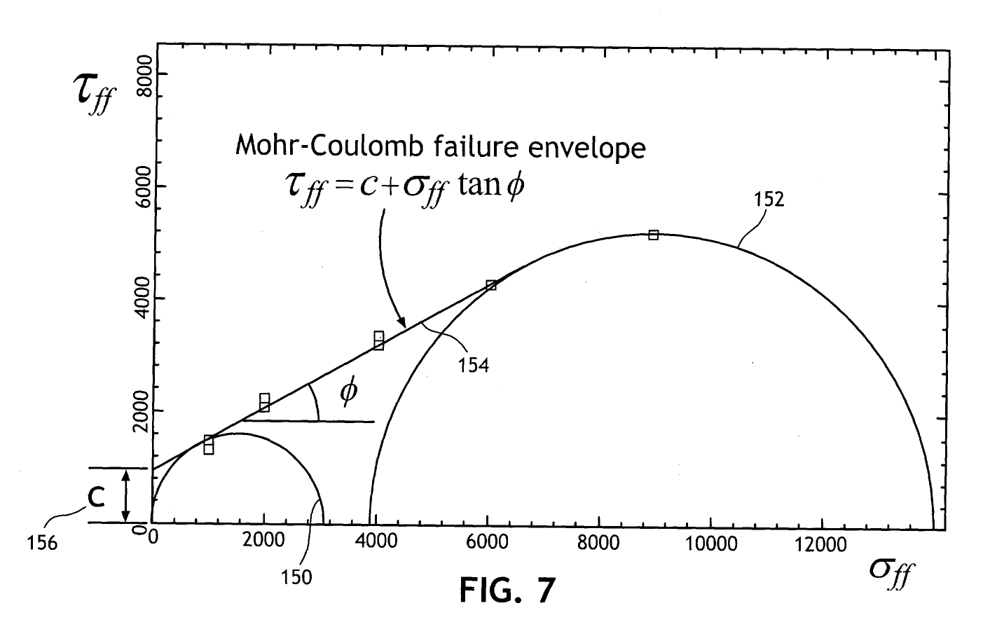

The friction angle and cohesion value of a composite bed are important properties that may affect the ability of the superabsorbent material to move or expand within the absorbent composite matrix. As discussed above in the Overview section, friction angle and cohesion comes from Mohr-Coulomb failure theory, and the tangent of the friction angle is equivalent to the traditional coefficient of static friction. A smaller friction angle may indicate less contact friction between the superabsorbent material and the surrounding matrix, and a greater ability for the superabsorbent material to rearrange within the matrix during swelling so that the superabsorbent material may retain a greater portion of the free swell absorbent capacity. Also, a smaller friction angle may promote failure (i.e., movement between, for example, swollen particles of superabsorbent material; or movement between a swollen particle of superabsorbent material and the surrounding fiber matrix; or, movement between individual fibers in contact with one another; etc.) at lower levels of stress buildup, thereby reducing losses in porosity and/or permeability in an absorbent composite. Cohesion equates to the shear stress at failure at a zero applied normal stress. A lower cohesion value may also promote failure as described above. In effect, a lower cohesion value means that the Mohr-Coulomb failure line is shifted downward on a plot of shear stress versus normal stress (such as those depicted in FIGS. 6 and 7).

The state of failure between the surfaces of the superabsorbent material and the surrounding components (e.g., fiber) allows the superabsorbent material to rearrange within composite. As indicated in the Overview Section, Mohr circles may be used to describe the state of stress of a material, such as a dry or wet fiber bed or absorbent composite or porous medium. FIG. 7 shows representative Mohr circles 150 and 152 for a typical composite bed. The larger Mohr circle 152 represents a situation where some pre-consolidation stress is imposed on the composite, and the smaller Mohr circle 150 represents the situation where some major principal stress exists anywhere in the composite while the minor principle stress is zero. Although not shown in FIG. 7, Mohr circles are produced at each applied normal stress. The state of failure for a fiber bed, gel bed, or composite bed is described by the set of Mohr circles at failure which together define a Mohr failure envelope. The Mohr failure envelope is often very close to linear, shown in FIG. 7 as line 154, and represents the shear stress at failure, on the failure plane, versus the normal stress acting on the same plane. The linearized failure envelope 154, often referred to as the Mohr-Coulomb failure criterion, may be represented mathematically by the formula:

_{τff}=c+σ_{ff}(tan φ)

where τ_{ff} is shear stress, c is the effective cohesion constant, σ_{ff} is normal stress, and φ is the friction angle of a material, such as a fiber bed, gel bed, or composite bed. The effective cohesion value is represented on the graph by value 156 and pertains to the cohesion of a fiber bed, gel bed, or composite bed.

The friction angle and effective cohesion constant (or cohesion value) of a composite of the present invention may be determined using various methods used in fields such as soil mechanics. Useful instruments for determining composite-bed friction angle include triaxial shear measurement instruments, such as a Sigma-1, available from GeoTac, Houston, Tex., or ring shear testers such as the Jenike-Shulze Ring Shear Tester, available from Jenike & Johanson, Inc., Wesfford, Mass.

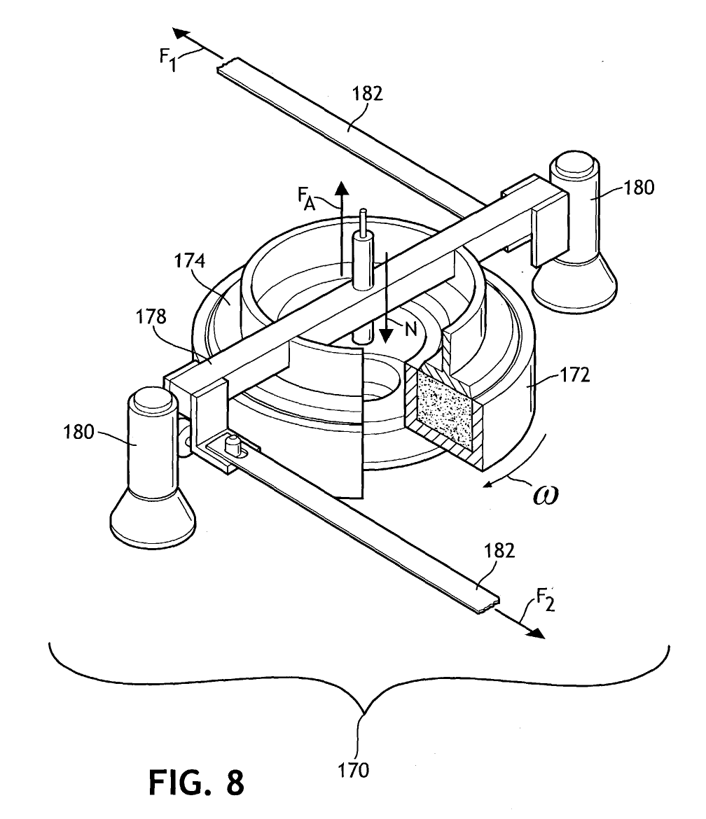

FIG. 8 shows a partial cut-away schematic of a Jenike-Shulze Ring Shear Tester, designated as reference numeral 170. The ring shear tester 170 has a ring shear cell 172 connected to a motor (not shown) that may rotate the ring shear cell 172 in direction ω. The ring shear cell 172 and lid 174 contain the composite (or other) bed 176 to be tested. The lid 174 is not fixed to the ring shear cell 172 and the crossbeam 178 crosses the lid 174 and connects two guiding rollers 180 and two tie rods 182 to lid 174. For measuring a composite bed of wet fiber and superabsorbent 176 the composite is wetted outside the ring shear cell 172 and placed in the ring shear cell 172. Of course this step is omitted when the friction angle and cohesion of a dry composite bed is being determined (Note: “dry” does not mean that all water is absent from the composite; some water will be present, even in a dry composite, at ambient conditions—e.g., about 2 to about 5% moisture based on the oven-dry weight of the composite. Oven-dry weight of a composite typically refers to the weight of the composite after the composite has been dried in an oven at 105 degrees Celsius.) A predetermined force N may be placed upon the lid 174, and therefore on the composite bed 176, by a weight (not shown). A counterweight system (not shown) may be engaged to test at lower normal pressure. As the ring shear cell 172 rotates in direction ω by the computer controlled motor (not shown) a shear force is placed on the composite bed 176 contacting the ring shear cell 172. An instrument connected to the tie rods 182 measures the forces F1 and F2, which are used to determine the shear stress at failure (for the given applied normal stress at which the test is conducted) of the composite bed 176. The cohesion value corresponds to the shear stress at failure for an applied normal stress of zero.

Composites having a low composite-bed friction angle and/or composite-bed cohesion value may be useful in absorbent products. Given that the amount of superabsorbent material relative to the amount of fiber in a composite affects the identity of the inter-particle interactions that predominate (e.g., fiber-fiber interactions; fiber-superabsorbent interactions; or, superabsorbent-superabsorbent interactions), suitable composite-bed properties may change with changing ratios of superabsorbent material to fiber. Generally, an increase in the dosage of superabsorbent material (here defined as the dry weight of superabsorbent in a composite divided by the dry weight of the composite—with the dry weight of the composite equaling the sum of the dry weight of fiber and the dry weight of superabsorbent when superabsorbent and fiber are the only ingredients making up the composite) corresponds to a decrease in friction angle, other factors being the same (e.g., degree of swelling for the superabsorbent).

Accordingly, in one embodiment of the present invention, a composite comprises a superabsorbent material and a fibrous matrix containing the superabsorbent material, wherein the superabsorbent dosage level is between about 20 and about 40 percent, wherein the composite-bed friction angle is less than about 30 degrees when composite-bed friction angle is evaluated using a composite bed swollen for one hour in a 20%-by-weight NaCl solution under an external load of 2,000 Pascals, and wherein the composite-bed friction angle is less than 30 degrees when composite-bed friction angle is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 20 and about 40 percent, wherein the composite-bed friction angle is less than about 26 degrees when composite-bed friction angle is evaluated using a composite bed swollen for one hour in a 20%-by-weight NaCl solution under an external load of 2,000 Pascals, and wherein the composite-bed friction angle is less than 26 degrees when composite-bed friction angle is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 20 and about 40 percent, wherein the composite-bed friction angle is less than about 20 degrees when composite-bed friction angle is evaluated using a composite bed swollen for one hour in a 20%-by-weight NaCl solution under an external load of 2,000 Pascals, and wherein the composite-bed friction angle is less than 20 degrees when composite-bed friction angle is evaluated using a composite bed swollen, under an external load of 2,000 Pascals, for one hour in a NaCl solution having a NaCl concentration less than 20% by weight.

It should be noted for these and other disclosed embodiments where swelling for one hour under a 2,000 Pascals load with a 20%-by-weight NaCl (or less-than-20%-by-weight NaCl) solution is referred to, that the external load of 2,000 Pascals refers to a load imposed on the composite bed when the composite bed is swollen with 20%-by-weight NaCl (or less-than-20%-by-weight NaCl) solution. Of course when the composite-bed friction angle and composite-bed cohesion value are measured on the resulting, swollen composite bed in a ring-shear tester as discussed herein, the applied normal load or stress varies from zero to some non-zero value.

In another embodiment of the present invention, a composite comprises a superabsorbent material and a fibrous matrix containing the superabsorbent material, wherein the superabsorbent dosage level is between about 40 and about 60 percent, wherein the composite-bed friction angle is less than about 27 degrees when composite-bed friction angle is evaluated using a composite bed swollen for one hour in a 20%-by-weight NaCl solution under an external load of 2,000 Pascals, and wherein the composite-bed friction angle is less than 27 degrees when composite-bed friction angle is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 40 and about 60 percent, wherein the composite-bed friction angle is less than about 22 degrees when composite-bed friction angle is evaluated using a composite bed swollen for one hour in a 20%-by-weight NaCl solution under an external load of 2,000 Pascals, and wherein the composite-bed friction angle is less than 22 degrees when composite-bed friction angle is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 40 and about 60 percent, wherein the composite-bed friction angle is less than about 17 degrees when composite-bed friction angle is evaluated using a composite bed swollen for one hour in a 20%-by-weight NaCl solution under an external load of 2,000 Pascals, and wherein the composite-bed friction angle is less than 17 degrees when composite-bed friction angle is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 60 and about 80 percent, wherein the composite-bed friction angle is less than about 25 degrees when composite-bed friction angle is evaluated using a composite bed swollen for one hour in a 20%-by-weight NaCl solution under an external load of 2,000 Pascals, and wherein the composite-bed friction angle is less than 25 degrees when composite-bed friction angle is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 60 and about 80 percent, wherein the composite-bed friction angle is less than about 20 degrees when composite-bed friction angle is evaluated using a composite bed swollen for one hour in a 20%-by-weight NaCl solution under an external load of 2,000 Pascals, and wherein the composite-bed friction angle is less than 20 degrees when composite-bed friction angle is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 60 and about 80 percent, wherein the composite-bed friction angle is less than about 15 degrees when composite-bed friction angle is evaluated using a composite bed swollen for one hour in a 20%-by-weight NaCl solution under an external load of 2,000 Pascals, and wherein the composite-bed friction angle is less than 15 degrees when composite-bed friction angle is evaluated using a composite bed swollen, under an external load of 2,000 Pascals, for one hour in a NaCl solution having a NaCl concentration less than 20% by weight.

The present invention also encompasses each of the embodiments described in the preceding ten paragraphs, wherein the composite-bed cohesion value is less than about 10,000 Pascals, suitably less than about 5,000 Pascals, particularly less than about 2,500 Pascals, and more particularly less than about 1,000 Pascals, with composite-bed cohesion values evaluated using a composite bed swollen for one hour in a 20%-by-weight NaCl solution under an external load of 2,000 Pascals.

In another embodiment of the present invention, a composite comprises a superabsorbent material and a fibrous matrix containing the superabsorbent material, wherein the superabsorbent dosage level is between about 20 and about 80 percent, wherein the composite-bed cohesion value is equal to or less than about 10,000 Pascals when composite-bed cohesion value is evaluated using a composite bed swollen for one hour in a 20%-by-weight NaCl solution under an external load of 2,000 Pascals, and wherein the composite-bed cohesion value is equal to or less than 10,000 Pascals when composite-bed cohesion value is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 20 and about 80 percent, wherein the composite-bed cohesion value is equal to or less than about 5,000 Pascals when composite-bed cohesion value is evaluated using a composite bed swollen for one hour in a 20%-by-weight NaCl solution under an external load of 2,000 Pascals, and wherein the composite-bed cohesion value is equal to or less than 5,000 Pascals when composite-bed cohesion value is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 20 and about 80 percent, wherein the composite-bed cohesion value is equal to or less than about 2,500 Pascals when composite-bed cohesion value is evaluated using a composite bed swollen for one hour in a 20%-by-weight NaCl solution under an external load of 2,000 Pascals, and wherein the composite-bed cohesion value is equal to or less than 2,500 Pascals when composite-bed cohesion value is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 20 and about 80 percent, wherein the composite-bed cohesion value is equal to or less than about 1,200 Pascals when composite-bed cohesion value is evaluated using a composite bed swollen for one hour in a 20%-by-weight NaCl solution under an external load of 2,000 Pascals.

The embodiments described in the preceding paragraphs may be prepared using fiber having controlled fiber-bed friction angles and/or controlled fiber-bed cohesion values; superabsorbent material having controlled gel-bed friction angles and/or controlled gel-bed cohesion values; or both. Such fibers and superabsorbents are described in co-pending applications identified, and incorporated by reference in their entirety in a manner consistent herewith, in the Background section above. When employed in an absorbent composite, low friction angle and/or cohesion value ingredients provide for a composite having the properties recited above. Such composites are less susceptible to the build up of large, local stresses occurring in the composite. For example, in an absorbent composite employing both a low gel-bed friction angle superabsorbent material and a low fiber-bed friction angle fiber, the ingredients help reduce the local stresses between the superabsorbent materials and the surrounding fiber matrix components, which may allow the superabsorbent material structures to rearrange within the voids of an absorbent composite matrix more easily. The low friction angle ingredients may allow for the superabsorbent materials to obtain a greater portion of their free swell absorbent capacity. In addition, permeability is generally maintained at suitable values because the development of higher internal stresses is alleviated. As indicated above, the buildup of stresses may result in additional compression of pore space.

In another embodiment of the present invention, ingredients having a high friction angle and/or cohesion are useful in an absorbent composite which is in a highly swollen state and/or in a high porosity state.

When an absorbent composite has high porosity and/or is in a highly swollen state, high friction angle and/or cohesion value ingredients may slow and/or inhibit rearranging within the absorbent composite matrix due to sheer failure and/or collapse. Slowing and/or inhibiting the rearrangement of, for example, superabsorbent material may maintain an open composite structure, if desired, thereby maintaining a desirable absorbent composite permeability. High friction angle and/or cohesion value ingredients may be particularly suitable for maintaining highly open structures when a load is subsequently applied. High fiber-bed friction-angle and/or fiber-bed cohesion-value fibers are described in co-pending applications identified, and incorporated by reference in their entirety in a manner consistent herewith, in the Background section above. Similarly, high gel-bed friction-angle superabsorbent materials are described in U.S. Provisional Patent Application Serial No. 60/399,794, entitled “Superabsorbent Materials Having High, Controlled Gel-Bed Friction Angles and Composites Made From The Same,” filed on Jul. 30, 2002 (as stated above, this co-pending application is incorporated by reference).

Accordingly, in one embodiment of the present invention, a composite comprises a superabsorbent material and a fibrous matrix containing the superabsorbent material, wherein the superabsorbent dosage level is between about 20 and about 40 percent, wherein the composite-bed friction angle is more than about 39 degrees when composite-bed friction angle is evaluated using a composite bed swollen for one hour in a 5%-by-weight NaCl solution under an external load of 2,000 Pascals, and wherein the composite-bed friction angle is more than 39 degrees when composite-bed friction angle is evaluated using a composite bed swollen, under an external load of 2,000 Pascals, for one hour in a NaCl solution having a NaCl concentration less than 5% by weight.

In another embodiment of the present invention, a composite comprises a superabsorbent material and a fibrous matrix containing the superabsorbent material, wherein the superabsorbent dosage level is between about 20 and about 40 percent, wherein the composite-bed friction angle is more than about 42 degrees when composite-bed friction angle is evaluated using a composite bed swollen for one hour in a 5%-by-weight NaCl solution under an external load of 2,000 Pascals, and wherein the composite-bed friction angle is more than about 42 degrees when composite-bed friction angle is evaluated using a composite bed swollen, under an external load of 2,000 Pascals, for one hour in a NaCl solution having a NaCl concentration less than 5% by weight.

In another embodiment of the present invention, a composite comprises a superabsorbent material and a fibrous matrix containing the superabsorbent material, wherein the superabsorbent dosage level is between about 20 and about 40 percent, wherein the composite-bed friction angle is more than about 46 degrees when composite-bed friction angle is evaluated using a composite bed swollen for one hour in a 5%-by-weight NaCl solution under an external load of 2,000 Pascals, and wherein the composite-bed friction angle is more than about 46 degrees when composite-bed friction angle is evaluated using a composite bed swollen, under an external load of 2,000 Pascals, for one hour in a NaCl solution having a NaCl concentration less than 5% by weight.

It should be noted for these and other disclosed embodiments where swelling for one hour under a 2,000 Pascals load with a 5%-by-weight NaCl solution (or lower) is referred to, that the external load of 2,000 Pascals refers to a load imposed on the composite bed when the composite bed is swollen with a 5%-by-weight NaCl solution, or with a solution having a lower NaCl concentration. Of course when the composite-bed friction angle and composite-bed cohesion value are measured on the resulting, swollen composite bed in a ring-shear tester as discussed herein, the applied normal load or stress varies from zero to some non-zero value.

In another embodiment of the present invention, a composite comprises a superabsorbent material and a fibrous matrix containing the superabsorbent material, wherein the superabsorbent dosage level is between about 40 and about 60 percent, wherein the composite-bed friction angle is more than about 35 degrees when composite-bed friction angle is evaluated using a composite bed swollen for one hour in a 5%-by-weight NaCl solution under an external load of 2,000 Pascals and wherein the composite-bed friction angle is more than about 35 degrees when composite-bed friction angle is evaluated using a composite bed swollen, under an external load of 2,000 Pascals, for one hour in a NaCl solution having a NaCl concentration less than 5% by weight.

In another embodiment of the present invention, a composite comprises a superabsorbent material and a fibrous matrix containing the superabsorbent material, wherein the superabsorbent dosage level is between about 40 and about 60 percent, wherein the composite-bed friction angle is more than about 38 degrees when composite-bed friction angle is evaluated using a composite bed swollen for one hour in a 5%-by-weight NaCl solution under an external load of 2,000 Pascals, and wherein the composite-bed friction angle is more than about 38 degrees when composite-bed friction angle is evaluated using a composite bed swollen, under an external load of 2,000 Pascals, for one hour in a NaCl solution having a NaCl concentration less than 5% by weight.

In another embodiment of the present invention, a composite comprises a superabsorbent material and a fibrous matrix containing the superabsorbent material, wherein the superabsorbent dosage level is between about 40 and about 60 percent, wherein the composite-bed friction angle is more than about 42 degrees when composite-bed friction angle is evaluated using a composite bed swollen for one hour in a 5%-by-weight NaCl solution under an external load of 2,000 Pascals, and wherein the composite-bed friction angle is more than about 42 degrees when composite-bed friction angle is evaluated using a composite bed swollen, under an external load of 2,000 Pascals, for one hour in a NaCl solution having a NaCl concentration less than 5% by weight.

In another embodiment of the present invention, a composite comprises a superabsorbent material and a fibrous matrix containing the superabsorbent material, wherein the superabsorbent dosage level is between about 60 and about 80 percent, wherein the composite-bed friction angle is more than about 33 degrees when composite-bed friction angle is evaluated using a composite bed swollen for one hour in a 5%-by-weight NaCl solution under an external load of 2,000 Pascals, and wherein the composite-bed friction angle is more than about 33 degrees when composite-bed friction angle is evaluated using a composite bed swollen, under an external load of 2,000 Pascals, for one hour in a NaCl solution having a NaCl concentration less than 5% by weight.

In another embodiment of the present invention, a composite comprises a superabsorbent material and a fibrous matrix containing the superabsorbent material, wherein the superabsorbent dosage level is between about 60 and about 80 percent, wherein the composite-bed friction angle is more than about 35 degrees when composite-bed friction angle is evaluated using a composite bed swollen for one hour in a 5%-by-weight NaCl solution under an external load of 2,000 Pascals, and wherein the composite-bed friction angle is more than about 35 degrees when composite-bed friction angle is evaluated using a composite bed swollen, under an external load of 2,000 Pascals, for one hour in a NaCl solution having a NaCl concentration less than 5% by weight.

In another embodiment of the present invention, a composite comprises a superabsorbent material and a fibrous matrix containing the superabsorbent material, wherein the superabsorbent dosage level is between about 60 and about 80 percent, wherein the composite-bed friction angle is more than about 40 degrees when composite-bed friction angle is evaluated using a composite bed swollen for one hour in a 5%-by-weight NaCl solution under an external load of 2,000 Pascals, and wherein the composite-bed friction angle is more than about 40 degrees when composite-bed friction angle is evaluated using a composite bed swollen, under an external load of 2,000 Pascals, for one hour in a NaCl solution having a NaCl concentration less than 5% by weight.

The present invention also encompasses each of the embodiments described in the preceding ten paragraphs, wherein the composite-bed cohesion value is more than about 100 Pascals, suitably more than about 500 Pascals, particularly more than about 1000 Pascals, and more particularly more than about 2,500 Pascals, with composite-bed cohesion values evaluated using a composite bed swollen for one hour in a 5%-by-weight NaCl solution under an external load of 2,000 Pascals.

In another embodiment of the present invention, a composite comprises a superabsorbent material and a fibrous matrix containing the superabsorbent material, wherein the superabsorbent dosage level is between about 20 and about 80 percent, wherein the composite-bed cohesion value is equal to or greater than about 4,500 Pascals when composite-bed cohesion value is evaluated using a composite bed swollen for one hour in a 20%-by-weight NaCl solution under an external load of 2,000 Pascals.

In another embodiment of the present invention, a composite comprises a superabsorbent material and a fibrous matrix containing the superabsorbent material, wherein the superabsorbent dosage level is between about 20 and about 80 percent, wherein the composite-bed cohesion value is equal to or greater than about 5,500 Pascals when composite-bed cohesion value is evaluated using a composite bed swollen for one hour in a 20%-by-weight NaCl solution under an external load of 2,000 Pascals.

In another embodiment of the present invention, a composite comprises a superabsorbent material and a fibrous matrix containing the superabsorbent material, wherein the superabsorbent dosage level is between about 20 and about 80 percent, wherein the composite-bed cohesion value is equal to or greater than about 6,500 Pascals when composite-bed cohesion value is evaluated using a composite bed swollen for one hour in a 20%-by-weight NaCl solution under an external load of 2,000 Pascals.

In another embodiment of the present invention, a composite comprises a superabsorbent material and a fibrous matrix containing the superabsorbent material, wherein the superabsorbent dosage level is between about 20 and about 80 percent, wherein the composite-bed cohesion value is equal to or greater than about 3,000 Pascals when composite-bed cohesion value is evaluated using a composite bed swollen for one hour in a 5%-by-weight NaCl solution under an external load of 2,000 Pascals.

In another embodiment of the present invention, a composite comprises a superabsorbent material and a fibrous matrix containing the superabsorbent material, wherein the superabsorbent dosage level is between about 20 and about 80 percent, wherein the composite-bed cohesion value is equal to or greater than about 4,000 Pascals when composite-bed cohesion value is evaluated using a composite bed swollen for one hour in a 5%-by-weight NaCl solution under an external load of 2,000 Pascals.

In another embodiment of the present invention, a composite comprises a superabsorbent material and a fibrous matrix containing the superabsorbent material, wherein the superabsorbent dosage level is between about 20 and about 80 percent, wherein the composite-bed cohesion value is equal to or greater than about 5,000 Pascals when composite-bed cohesion value is evaluated using a composite bed swollen for one hour in a 5%-by-weight NaCl solution under an external load of 2,000 Pascals.

Accordingly, in one embodiment of the present invention, a composite comprises a superabsorbent material and a fibrous matrix containing the superabsorbent material, wherein the superabsorbent dosage level is between about 20 and about 40 percent, wherein the composite-bed friction angle is less than about 30 degrees when composite-bed friction angle is evaluated using a composite bed swollen for one hour in a 20%-by-weight NaCl solution under an external load of 2,000 Pascals, and wherein the composite-bed friction angle is equal to or greater than about 30 degrees when composite-bed friction angle is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 20 and about 40 percent, wherein the composite-bed friction angle is less than about 26 degrees when composite-bed friction angle is evaluated using a composite bed swollen for one hour in a 20%-by-weight NaCl solution under an external load of 2,000 Pascals, and wherein the composite-bed friction angle is equal to or greater than about 26 degrees when composite-bed friction angle is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 20 and about 40 percent, wherein the composite-bed friction angle is less than about 20 degrees when composite-bed friction angle is evaluated using a composite bed swollen for one hour in a 20%-by-weight NaCl solution under an external load of 2,000 Pascals, and wherein the composite-bed friction angle is equal to or greater than about 20 degrees when composite-bed friction angle is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 40 and about 60 percent, wherein the composite-bed friction angle is less than about 27 degrees when composite-bed friction angle is evaluated using a composite bed swollen for one hour in a 20%-by-weight NaCl solution under an external load of 2,000 Pascals, and wherein the composite-bed friction angle is equal to or greater than about 27 degrees when composite-bed friction angle is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 40 and about 60 percent, wherein the composite-bed friction angle is less than about 22 degrees when composite-bed friction angle is evaluated using a composite bed swollen for one hour in a 20%-by-weight NaCl solution under an external load of 2,000 Pascals, and wherein the composite-bed friction angle is equal to or greater than 22 degrees when composite-bed friction angle is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 40 and about 60 percent, wherein the composite-bed friction angle is less than about 17 degrees when composite-bed friction angle is evaluated using a composite bed swollen for one hour in a 20%-by-weight NaCl solution under an external load of 2,000 Pascals, and wherein the composite-bed friction angle is equal to or greater than 17 degrees when composite-bed friction angle is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 60 and about 80 percent, wherein the composite-bed friction angle is less than about 25 degrees when composite-bed friction angle is evaluated using a composite bed swollen for one hour in a 20%-by-weight NaCl solution under an external load of 2,000 Pascals, and wherein the composite-bed friction angle is equal to or greater than 25 degrees when composite-bed friction angle is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 60 and about 80 percent, wherein the composite-bed friction angle is less than about 20 degrees when composite-bed friction angle is evaluated using a composite bed swollen for one hour in a 20%-by-weight NaCl solution under an external load of 2,000 Pascals, and wherein the composite-bed friction angle is equal to or greater than 20 degrees when composite-bed friction angle is evaluated using a composite bed swollen, under an external load of 2,000 Pascals, for one hour in a NaCl solution having a NaCl concentration less than 20% by weight.

In another embodiment of the present invention, a composite comprises a superabsorbent material and a fibrous matrix containing the superabsorbent material, wherein the superabsorbent dosage level is between about 60 and about 80 percent, wherein the composite-bed friction angle is less than about 15 degrees when composite-bed friction angle is evaluated using a composite bed swollen for one hour in a 20%-by-weight NaCl solution under an external load of 2,000 Pascals, and wherein the composite-bed friction angle is equal to or greater than 15 degrees when composite-bed friction angle is evaluated using a composite bed swollen, under an external load of 2,000 Pascals, for one hour in a NaCl solution having a NaCl concentration less than 20% by weight.

The present invention also encompasses each of the embodiments described in the preceding ten paragraphs, wherein the composite-bed cohesion value is less than about 10,000 Pascals, suitably less than about 5,000 Pascals, particularly less than about 2,500 Pascals, and more particularly less than about 1,000 Pascals, with composite-bed cohesion values evaluated using a composite bed swollen for one hour in a 20%-by-weight NaCl solution under an external load of 2,000 Pascals.

The additives, such as the friction angle increasing additives and friction angle reducing additives, which may alter the friction angle of superabsorbent materials, may be delivered either directly or indirectly to the superabsorbent. Direct delivery could occur through release from the superabsorbent material itself while indirect delivery could occur from fiber or some other component positioned within or adjacent the superabsorbent material and/or the absorbent composite. Furthermore, friction angle altering additives may be delivered gradually over some time period through release from any of the existing components present in the absorbent composite or as the result of some chemical reaction devised to release the friction angle altering additive at the most desirable moment. For example, the friction angle altering additive may be attached to the surface of the superabsorbent material or embedded within its interior, or it may be loaded onto and/or into some other component present in the absorbent composite, including but not limited to the fibrous material. The friction angle altering additive may be available immediately, leading to immediate alteration of the friction angle, or because of a chemical reaction or diffusion or some other mechanism, gradually alter the friction angle in the desired manner at some desired time. When using mixtures of polar and nonpolar compounds, such as friction angle or cohesion value altering additives, emulsifiers, and surfactants, the nonpolar compound may be present in a larger proportion than the polar compound.

It may be desirable to treat the superabsorbent material, the fiber and/or fibrous matrix, and/or other components that may be used in an absorbent composite with a friction angle altering additive, such as the friction angle reducing additive, the friction angle increasing additive and/or combinations thereof, to provide materials having desired initial friction angles. The material treated with the friction angle altering additive to provide a desired initial friction angle may then be treated with additional friction angle altering additives in accordance with the present invention.

Composites having controlled composite-bed friction angle and/or controlled composite-bed cohesion values may be incorporated into absorbent articles.

In accordance with one embodiment of the present invention, an absorbent composite may comprise a fibrous matrix and a water swellable, water insoluble superabsorbent material in combination with the fibrous matrix in a dosage level of between about 20 and about 40 percent. The absorbent composite may have a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals. The absorbent composite-bed friction angles may be substantially equal to or less than the first composite-bed friction angle. The first composite-bed friction angle may be about 30 degrees or less. In the alternative, the first composite-bed friction angle may be about 20 degrees or less. (The term “substantially” when used herein in regard with friction angle, means within +/− one degree. The term “substantially” when used herein in regard with cohesion value, means within +/− 100 Pascals.)

The absorbent composite may have a composite-bed cohesion value of about 10,000 Pascals or less when swollen in a NaCl solution having a NaCl concentration of about 20% by weight for one hour under an external load of 2,000 Pascals. The water swellable, water insoluble superabsorbent material may be selected from the group consisting essentially of natural materials, synthetic materials, modified natural materials, and combinations thereof.

The water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof. In the alternative, the water swellable, water insoluble superabsorbent material may further comprise a structure selected from the group consisting essentially of particles, fibers, flakes, spheres, and combinations thereof.

In accordance with another embodiment of the present invention, an absorbent composite may comprise a fibrous matrix and a water swellable, water insoluble superabsorbent material in combination with the fibrous matrix in a dosage level of between about 40 and about 60 percent. The absorbent composite may have a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals. The composite-bed friction angles may be substantially equal to or less than the first composite-bed friction angle. The first composite-bed friction angle may be about 27 degrees or less. In the alternative, the first composite-bed friction angle may be about 17 degrees or less.

The absorbent composite may have a composite-bed cohesion value of about 10,000 Pascals or less when swollen in a NaCl solution having a NaCl concentration of about 20% by weight for one hour under an external load of 2,000 Pascals. The water swellable, water insoluble superabsorbent material may be selected from the group consisting essentially of natural materials, synthetic materials, modified natural materials, and combinations thereof.

The water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof. The water swellable, water insoluble superabsorbent material may further comprise a structure selected from the group consisting essentially of particles, fibers, flakes, spheres, and combinations thereof.

In accordance with another embodiment of the present invention, an absorbent composite may comprise a fibrous matrix and a water swellable, water insoluble superabsorbent material in combination with the fibrous matrix in a dosage level of between about 60 and about 80 percent. The absorbent composite may have a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals. The composite-bed friction angles may be substantially equal to or less than the first composite-bed friction angle. The first composite-bed friction angle may be about 25 degrees or less. In the alternative, the first composite-bed friction angle may be about 15 degrees or less.

The absorbent composite may have a composite-bed cohesion value of about 10,000 Pascals or less when swollen in a NaCl solution having a NaCl concentration of about 20% by weight for one hour under an external load of 2,000 Pascals. The water swellable, water insoluble superabsorbent material may be selected from the group consisting essentially of natural materials, synthetic materials, modified natural materials, and combinations thereof.

The water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof. The water swellable, water insoluble superabsorbent material may further comprise a structure selected from the group consisting essentially of particles, fibers, flakes, spheres, and combinations thereof.

In accordance with another embodiment of the present invention, an absorbent composite may comprise a fibrous matrix and a water swellable, water insoluble superabsorbent material in combination with the fibrous matrix in a dosage level of between about 20 and about 80 percent. The absorbent composite having a first composite-bed cohesion value when swollen in a NaCl solution having a NaCl concentration of about 20% by weight for one hour under an external load of 2,000 Pascals and composite-bed cohesion values, when swollen in a NaCl solution having a NaCl concentration of less than about 20% by weight for one hour under an external load of 2,000 Pascals. The composite-bed cohesion values may be substantially equal to or less than the first composite-bed cohesion value. The first composite-bed cohesion value may be about 1,200 Pascals or less. In the alternative, the first composite-bed cohesion value may be about 500 Pascals or less.

The water swellable, water insoluble superabsorbent material may be selected from the group consisting essentially of natural materials, synthetic materials, modified natural materials, and combinations thereof. The water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof. The water swellable, water insoluble superabsorbent material may further comprise a structure selected from the group consisting essentially of particles, fibers, flakes, spheres, and combinations thereof.

In accordance with another embodiment of the present invention, an absorbent composite may comprise a fibrous matrix and a water swellable, water insoluble superabsorbent material in combination with the fibrous matrix in a dosage level of between about 20 and about 40 percent. The absorbent composite may have a first composite-bed friction angle when swollen in a NaCl solution having a NaCl concentration of about 5% by weight for one hour under an external load of 2,000 Pascals and composite-bed friction angles, when swollen in a NaCl solution having a NaCl concentration of less than about 5% by weight for one hour under an external load of 2,000 Pascals. The composite-bed friction angles may be substantially equal to or greater than the first composite-bed friction angle. The first composite-bed friction angle may be about 39 degrees or greater. In the alternative, the first composite-bed friction angle may be about 46 degrees or greater.

The absorbent composite may have a composite-bed cohesion value of about 100 Pascals or greater when swollen in a NaCl solution having a NaCl concentration of about 5% by weight for one hour under an external load of 2,000 Pascals. The water swellable, water insoluble superabsorbent material may be selected from the group consisting essentially of natural materials, synthetic materials, modified natural materials, and combinations thereof. The water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof. In the alternative, the water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, and combinations thereof. The water swellable, water insoluble superabsorbent material may further comprise a structure selected from the group consisting essentially of particles, fibers, flakes, spheres, and combinations thereof.

In accordance with another embodiment of the present invention, an absorbent composite may comprise a fibrous matrix and a water swellable, water insoluble superabsorbent material in combination with the fibrous matrix in a dosage level of between about 40 and about 60 percent. The absorbent composite may have a first composite-bed friction angle when swollen in a NaCl solution having a NaCl concentration of about 5% by weight for one hour under an external load of 2,000 Pascals and composite-bed friction angles, when swollen in a NaCl solution having a NaCl concentration of less than about 5% by weight for one hour under an external load of 2,000 Pascals. The composite-bed friction angles may be substantially equal to or greater than the first composite-bed friction angle. The first composite-bed friction angle may be about 35 degrees or greater. In the alternative, the first composite-bed friction angle may be about 42 degrees or greater.

The absorbent composite may have a composite-bed cohesion value of about 100 Pascals or greater when swollen in a NaCl solution having a NaCl concentration of about 5% by weight for one hour under an external load of 2,000 Pascals. The water swellable, water insoluble superabsorbent material may be selected from the group consisting essentially of natural materials, synthetic materials, modified natural materials, and combinations thereof.

The water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof. In the alternative, the water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, and combinations thereof. The water swellable, water insoluble superabsorbent material may further comprise a structure selected from the group consisting essentially of particles, fibers, flakes, spheres, and combinations thereof.

In accordance with another embodiment of the present invention, an absorbent composite may comprise a fibrous matrix and a water swellable, water insoluble superabsorbent material in combination with the fibrous matrix in a dosage level of between about 60 and about 80 percent. The absorbent composite may have a first composite-bed friction angle when swollen in a NaCl solution having a NaCl concentration of about 5% by weight for one hour under an external load of 2,000 Pascals and composite-bed friction angles, when swollen in a NaCl solution having a NaCl concentration of less than about 5% by weight for one hour under an external load of 2,000 Pascals. The composite-bed friction angles may be substantially equal to or greater than the first composite-bed friction angle. The first composite-bed friction angle may be about 33 degrees or greater. In the alternative, the first composite-bed friction angle may be about 40 degrees or greater.

The absorbent composite may have a composite-bed cohesion value of about 100 Pascals or greater when swollen in a NaCl solution having a NaCl concentration of about 5% by weight for one hour under an external load of 2,000 Pascals. The water swellable, water insoluble superabsorbent material may be selected from the group consisting essentially of natural materials, synthetic materials, modified natural materials, and combinations thereof.

The water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof. In the alternative, the water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, and combinations thereof. The water swellable, water insoluble superabsorbent material may further comprise a structure selected from the group consisting essentially of particles, fibers, flakes, spheres, and combinations thereof.

In accordance with another embodiment of the present invention, an absorbent composite may comprise a fibrous matrix and a water swellable, water insoluble superabsorbent material in combination with the fibrous matrix in a dosage level of between about 20 and about 80 percent. The absorbent composite may have a first composite-bed cohesion value when swollen in a NaCl solution having a NaCl concentration of about 20% by weight for one hour under an external load of 2,000 Pascals and composite-bed cohesion values, when swollen in a NaCl solution having a NaCl concentration of less than about 20% by weight for one hour under an external load of 2,000 Pascals. The composite-bed cohesion values may be substantially equal to or greater than the first composite-bed cohesion value. The first composite-bed friction angle may be about 4,500 Pascals or greater. In the alternative, the first composite-bed cohesion value may be about 6,500 Pascals or greater.

The water swellable, water insoluble superabsorbent material may be selected from the group consisting essentially of natural materials, synthetic materials, modified natural materials, and combinations thereof. The water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof. The water swellable, water insoluble superabsorbent material may further comprise a structure selected from the group consisting essentially of particles, fibers, flakes, spheres, and combinations thereof.

In accordance with another embodiment of the present invention, an absorbent composite may comprise a fibrous matrix and a water swellable, water insoluble superabsorbent material in combination with the fibrous matrix in a dosage level of between about 20 and about 80 percent. The absorbent composite may have a first composite-bed cohesion value when swollen in a NaCl solution having a NaCl concentration of about 5% by weight for one hour under an external load of 2,000 Pascals and composite-bed cohesion values, when swollen in a NaCl solution having a NaCl concentration of less than about 5% by weight for one hour under an external load of 2,000 Pascals. The composite-bed cohesion values may be substantially equal to or greater than the first composite-bed friction angle. The first composite-bed cohesion value may be about 3,000 Pascals or greater. In the alternative, the first composite-bed cohesion value may be about 5,000 Pascals or greater.

The water swellable, water insoluble superabsorbent material may be selected from the group consisting essentially of natural materials, synthetic materials, modified natural materials, and combinations thereof. The water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof. In the alternative, the water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, and combinations thereof. The water swellable, water insoluble superabsorbent material may further comprise a structure selected from the group consisting essentially of particles, fibers, flakes, spheres, and combinations thereof.

In accordance with another embodiment of the present invention, an absorbent composite may comprise a fibrous matrix and a water swellable, water insoluble superabsorbent material in combination with the fibrous matrix in a dosage level of between about 20 and about 40 percent. The absorbent composite may have a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals. The composite-bed friction angles may be substantially equal to or greater than the first composite-bed friction angle. The first composite-bed friction angle may be about 30 degrees or less. In the alternative, the first composite-bed friction angle may be about 20 degrees or less.

The absorbent composite may have a composite-bed cohesion value of about 10,000 Pascals or less when swollen in a NaCl solution having a NaCl concentration of about 20% by weight for one hour under an external load of 2,000 Pascals. The water swellable, water insoluble superabsorbent material may be selected from the group consisting essentially of natural materials, synthetic materials, modified natural materials, and combinations thereof.

The water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof. The water swellable, water insoluble superabsorbent material may further comprise a structure selected from the group consisting essentially of particles, fibers, flakes, spheres, and combinations thereof.

In accordance with another embodiment of the present invention, an absorbent composite may comprise a fibrous matrix and a water swellable, water insoluble superabsorbent material in combination with the fibrous matrix in a dosage level of between about 40 and about 60 percent. The absorbent composite may have a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals. The composite-bed friction angles may be substantially equal to or greater than the first composite-bed friction angle. The first composite-bed friction angle may be about 27 degrees or less. In the alternative, the first composite-bed friction angle may be about 17 degrees or less.

The absorbent composite may have a composite-bed cohesion value of about 10,000 Pascals or less when swollen in a NaCl solution having a NaCl concentration of about 20% by weight for one hour under an external load of 2,000 Pascals.

The water swellable, water insoluble superabsorbent material may be selected from the group consisting essentially of natural materials, synthetic materials, modified natural materials, and combinations thereof. The water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof. The water swellable, water insoluble superabsorbent material may further comprise a structure selected from the group consisting essentially of particles, fibers, flakes, spheres, and combinations thereof.

In accordance with another embodiment of the present invention, an absorbent composite may comprise a fibrous matrix and a water swellable, water insoluble superabsorbent material in combination with the fibrous matrix in a dosage level of between about 60 and about 80 percent. The absorbent composite may have a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals. The composite-bed friction angles may be substantially equal to or greater than the first composite-bed friction angle. The first composite-bed friction angle may be about 25 degrees or less. In the alternative, the first composite-bed friction angle may be about 15 degrees or less.

The absorbent composite may have a composite-bed cohesion value of about 10,000 Pascals or less when swollen in a NaCl solution having a NaCl concentration of about 20% by weight for one hour under an external load of 2,000 Pascals. The water swellable, water insoluble superabsorbent material may be selected from the group consisting essentially of natural materials, synthetic materials, modified natural materials, and combinations thereof.

The water swellable, water insoluble superabsorbent material may be selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof. The water swellable, water insoluble superabsorbent material may further comprise a structure selected from the group consisting essentially of particles, fibers, flakes, spheres, and combinations thereof.

### Friction Angle and Cohesion Value Determination

Test Procedure:

(Composite Swelling; and Friction angle and Cohesion Measurement):

Ring Shear Tester

Composite

Purpose:

To Calculate Friction Angle and Effective Cohesion Value from the Normal Force Applied and the Shear Force Used

Equation: τ=C+σ(tangentφ)

Variables:

| | | |
|---|---|---|
| τ | shear force needed for smooth movement | (tau) |
| C | effective cohesion value at 0 normal force | (or σ= 0) |
| σ | normal force applied (could also use “N” | (sigma) |
| φ | friction angle | (phi) |
| φlinear | Linear friction angle | (phi lin) |

File Labeling

Purpose:

File and Bulk Solids label should include the Composite Code number, the normal load ramp, Wet or Dry—type, and cell ring information

Example: Composite 1 (60%9543 40% NB416 600gsm) Wet—fully saturated in cell ring #2 with T1A normal load ramp

File name: A01W1TA2

| A | For Composite Code |
|---|---|
| 01 | Number of Composite Code |
| TA | For Load Ramp |
| W1 | Wet (from Wet or Dry) and type of wetting |
| 2 | Ring Cell Number |

Procedure:

Composite Preparation

1 Determine Fiber Type, Sap Type, Percentages, Basis Weight and Wet/Dry

2 Make Handsheets required at given Basis Weight Example 10×17in2@600 gsm

3 Cut Circular Ring shapes out of Handsheets—Dimensions: 34.61 in2

4 Collect Dry Weight in grams

5 For Dry Composite readings Skip to Step# 21, For Wet Composite readings go onto to Step#6

6 Place Sample into plastic soaking ring chamber, and place chamber into Fluid Box Reservoir

7 Place Ring Plate on top of sample in ring chamber (160.32 g)

8 Place Additional Weight onto Ring Plate to achieve 2,000 Pascals load (160.32+4548.92 g=47( Fill Fluid Box with 1 inch NaCl solution at required concentration,

9 and wait 60 minutes for soaking and swelling

10 Remove Weights and Pull out Ring Chamber (with sample and plate) from Fluid Box Reservoir

11 Wipe Assembly to keep from dripping

12 Flip Chamber/Sample/Plate quickly and place on top of 1 blotter

13 Push out Sample and Plate (now under sample) from Ring Chamber

14 Place 5+ blotters on top of sample and flip all—blotter/plate/sample/blotters

15 Remove top blotter (former bottom) and Ring Plate, sample just remains on blotters

16 Cover Sample with 5 new blotters, gently press only for contact

17 Allow for desorption—30 minutes

18 Flip sample/blotters and exchange wet top blotters for new dry ones

19 Allow for desorption on other side—30 minutes

20 Remove top blotters

21 Peal away forming tissue from sample with forceps-gently

22 Flip sample and peal away other forming tissue

23 Place sample into Ring Cell #2

24 Now either finish Computer Set-up or Go onto Running Test

NOTE:

*During Fiber Preparation Step 10 do Computer Set-up, Calibration must be done before Step

Computer Set-up and Calibration

1 Turn on Computer and Ring Shear Tester—wait 30 minutes

2 After 30 minutes, Press Start Icon and up to Programs-Press Select Miss.

3 DOS

4 When in MS DOS after C:>WINDOWS>, write in cd., then enter

5 After prompt: C:>, write in: cd rsv, then enter

6 After prompt: RSV:>, write in: rstctrl, then enter

7 It will tell you to switch on ring shear tester—confirm that it is still on, press space bar

8 Tester will do some initiation steps-wait

9 Computer will mention “check offset values . . . ”, If the same press Y for Yes

10 Place empty ring shear cell with lid on to tester and connect hanger, press space bar

11 It will test upper limit, wait, press space bar no tie rods here

12 It will test lower limit, wait, press space bar no tie rods here

13 Note that there are no tie rods on yet, press space bar

14 Press F1 for “TESTS”

15 Press F1 for “Flow Properties”

16 Press F4 for “Read Settings from Control File”

See File

17 At “Bulk Solids” enter name of file/experiment, press enter Labeling ex:A01W1TA2

18 At “Order” enter in information of sample/test, press enter ex. Code 1 Wet T1A

19 At “Ring Shear #” enter Cell # ex. 2

20 At “Total Mass” stop and finish Composite Preparation if necessary

21 Go on to Running Test

Running Test: Ring Shear Tester

1 Weigh Filled Ring Shear Cell, from Composite Preparation Step 21/22* Record

2 Weight example 3338.5

3 Insert Filled Ring Shear Cell onto the Tester, click into place

4 On computer, at “Total Mass” enter the recorded weight, press enter

5 For presettings, press Y for Yes

6 At “Control File Prefix:: enter T1A, then enter

7 It will give a range, wait

8 It will ask “Start Measuring with These Settings”, enter Y for Yes

9 It will say to put the bottom ring on, the top on (evenly), connect hanger—forgot the weight confirm bottom is on, put on top, connect counter weight, connect hanger, press space bar

10 It will ask you to confirm the weight is on, confirm and press space bar

11 It will ask you to confirm that the tie rods are not on, confirm and press space bar

12 It will recheck force values, when prompt—press space bar

13 At prompt, place tie rods on, place R and L tie rods, adjust center (if need), press space bar

14 Test starts running (1-2 hours total), It will start with the pre-shearing

15 Press F2 to change to Normal Velocity

16 Record the Sample Mass number example 124.40

16 When the pre-shear force is at equilibrium (flat line) it should automatically change to the first normal force 500, and then continue on with testing each normal force

17 After last normal force finishes, it will say test complete

18 Record values phiSF (degrees) and FC[Pa] press space bar and it will show values, press space bar again

19 It will ask you to save file, enter Y for Yes enter file name—should be the same as the “Bulk Solids” label, press space bar

20 It will ask you to store data, enter Y for Yes

23 To do another test select F1 for “Flow properties” and repeat from step 15 in Computer set up,

24 To leave the program press Esc for main menu

25 Press Esc, to exit program

26 Press Y for Yes to terminate

27 Close window for DOS, and press Start and up to Shut down

### EXAMPLES

To demonstrate aspects of the present invention, fibers NB416, available from Weyerhaeuser, a business having offices in Federal Way, Wash., and Sulfatate HJ, available from Rayonier, a business having offices in Jesup, Ga.; were treated to alter the airformed composite friction angle and airformed composite cohesion. All airformed composites (which included superabsorbent material FAVOR® 9543SXM, available from Stockhausen, Inc) were made to a basis weight about 600 grams per square meter with densities about 0.10 grams per cubic centimeter. Those airformed composites that included treated fiber were made to basis weight about 600 grams per square meter with densities about 0.10 grams per cubic centimeter based upon dry untreated components (fiber and/or sap) only; they were adjusted for the treatment presence.

Treatments used within these examples were either sprayed onto or printed onto both sides of the fiber roll board to achieve desired add on levels. The fibers were then fiberized with a Kamas fiberizer commercially available from Kamas Industri AB, located at Vellinge, Sweden, at settings that gave a 95 or more percentage of fiberization. The fiberized treated fibers were used to make airformed fiber-beds and airformed composites.

### Control 1

An air-formed composite was made from 60% weight (on dry basis) superabsorbent material, untreated FAVOR® SXM 9543 (available from Stockhausen, Inc., a business having offices in Greensboro, N.C.) and 40% weight (on dry basis) fluff fiber designated as NB416 (available from Weyerhaeuser, a business having offices in Federal Way, Wash.). The composite was swollen, following the method given above, in solutions of 20%, 10%, 5% and 0.9% by weight aqueous NaCl solution. The composite-bed friction angles and composite-bed cohesion values were measured as described in the procedure given above. The composite-bed friction angle and composite-bed cohesion value of the swollen composite were found to be 30 degrees and 1653 Pascals, 26 degrees and 978 Pascals, 26 degrees and 1043 Pascals, and 19 degrees and 592 Pascals, respectively, which are summarized in Table 1 and Table 2.

### Control 2

An air-formed composite was made from 40% weight (on dry basis) superabsorbent material, untreated FAVOR® SXM 9543 (from Control 1) and 60% weight (on dry basis) fluff fiber designated as NB416 (from Control 1). The composite was swollen, following the method given above, in solutions of 20%, 10%, 5% and 0.9% by weight aqueous NaCl solution. The composite-bed friction angles and composite-bed cohesion values were measured as described in the procedure given above. The composite-bed friction angle and composite-bed cohesion value of the swollen composite were found to be 32 degrees and 1426 Pascals, 25 degrees and 768 Pascals, 26 degrees and 760 Pascals, and 21 degrees and 469 Pascals, respectively, which are summarized in Table 1 and Table 2.

### Control 3

An air-formed composite was made from 20% weight (on dry basis) superabsorbent material, untreated FAVOR® SXM 9543 (from Control 1) and 80% weight (on dry basis) fluff fiber designated as NB416 (from Control 1). The composite was swollen, following the method given above, in solutions of 20%, 10%, 5% and 0.9% by weight aqueous NaCl solution. The composite-bed friction angles and composite-bed cohesion values were measured as described in the procedure given above. The composite-bed friction angle and composite-bed cohesion value of the swollen composite were found to be 37 degrees and 2589 Pascals, 40 degrees and 2077 Pascals, 36 degrees and 1249 Pascals, and 24 degrees and 902 Pascals, respectively, which are summarized in Table 1 and Table 2.

### Control 4

An air-formed composite was made from 60% weight (on dry basis) superabsorbent material, untreated FAVOR® SXM 9543 (from Control 1) and 40% weight (on dry basis) fluff fiber designated as Sulfatate HJ (available from Rayonier, a business having offices in Jesup, Ga.). The composite was swollen, following the method given above, in solutions of 20%, 10%, 5% and 0.9% by weight aqueous NaCl solution. The composite-bed friction angles and composite-bed cohesion values were measured as described in the procedure given above. The composite-bed friction angle and composite-bed cohesion value of the swollen composite were found to be 31 degrees and 1935 Pascals, 29 degrees and 1768 Pascals, 26 degrees and 1503 Pascals, and 21 degrees and 619 Pascals, respectively, which are summarized in Table 1 and Table 2.

### Control 5

An air-formed composite was made from 40% weight (on dry basis) superabsorbent material, untreated FAVOR® SXM 9543 (from Control 1) and 60% weight (on dry basis) fluff fiber designated as Sulfatate HJ (from Control 4). The composite was swollen, following the method given above, in solutions of 20%, 10%, 5% and 0.9% by weight aqueous NaCl solution. The composite-bed friction angles and composite-bed cohesion values were measured as described in the procedure given above. The composite-bed friction angle and composite-bed cohesion value of the swollen composite were found to be 33 degrees and 2123 Pascals, 29 degrees and 1846 Pascals, 27 degrees and 1085 Pascals, and 22 degrees and 873 Pascals, respectively, which are summarized in Table 1 and Table 2.

### Control 6

An air-formed composite was made from 20% weight (on dry basis) superabsorbent material, untreated FAVOR® SXM 9543 (from Control 1) and 80% weight (on dry basis) fluff fiber designated as Sulfatate HJ (from Control 4). The composite was swollen, following the method given above, in solutions of 20%, 10%, 5% and 0.9% by weight aqueous NaCl solution. The composite-bed friction angles and composite-bed cohesion values were measured as described in the procedure given above. The composite-bed friction angle and composite-bed cohesion value of the swollen composite were found to be 39 degrees and 4063 Pascals, 39 degrees and 2970 Pascals, 38 degrees and 2813 Pascals, and 33 degrees and 1627 Pascals, respectively, which are summarized in Table 1 and Table 2.

TABLE 1
Composite Friction Angles (in degrees) with various solutions

| | 20% Saline | 10% Saline | 5% Saline | 0.9% Saline |
|---|---|---|---|---|
| Code | Solution | Solution | Solution | Solution |
| Control 1 | 30 | 26 | 26 | 19 |
| (60% 9543/40% | | | | |
| NB416) | | | | |
| Control 2 | 32 | 25 | 26 | 21 |
| (40% 9543 60% | | | | |
| NB416) | | | | |
| Control 3 | 37 | 40 | 36 | 24 |
| (20% 9543/80% | | | | |
| NB416) | | | | |
| Control 4 | 31 | 29 | 26 | 21 |
| (60% 9543/40% | | | | |
| Sulfatate HJ) | | | | |
| Control 5 | 33 | 29 | 27 | 22 |
| (40% 9543/60% | | | | |
| Sulfatate HJ) | | | | |
| Control 6 | 39 | 39 | 38 | 33 |
| (20% 9543/80% | | | | |
| Sulfatate HJ) | | | | |

TABLE 2
Composite Cohesion (in Pascals) with various solutions

| | 20% Saline | 10% Saline | 5% Saline | 0.9% Saline |
|---|---|---|---|---|
| Code | Solution | Solution | Solution | Solution |
| Control 1 | 1653 | 978 | 1043 | 592 |
| (60% 9543/40% | | | | |
| NB416) | | | | |
| Control 2 | 1426 | 768 | 760 | 469 |
| (40% 9543/60% | | | | |
| NB416) | | | | |
| Control 3 | 2589 | 2077 | 1249 | 902 |
| (20% 9543/80% | | | | |
| NB416) | | | | |
| Control 4 | 1935 | 1768 | 1503 | 619 |
| (60% 9543/40% | | | | |
| Sulfatate HJ) | | | | |
| Control 5 | 2123 | 1846 | 1085 | 873 |
| (40% 9543/60% | | | | |
| Sulfatate HJ) | | | | |
| Control 6 | 4063 | 2970 | 2813 | 1627 |
| (20% 9543/80% | | | | |
| Sulfatate HJ) | | | | |

### Example 1

An air-formed composite was made from 20% weight (on dry basis) superabsorbent material, untreated FAVOR® SXM 9543 (from Control 1) and 80% weight (on dry basis) fluff fiber of NB416 (from Control 1) blended with T255, a synthetic KoSa Celbond® bicomponent fiber available from KoSa, at a ratio of 0.5 grams NB416 and 0.5 grams of T255 per 1.0 grams of fiber. The composite was swollen, following the method given above, in solutions of 20%, 10%, 5% and 0.9% by weight aqueous NaCl solution. The composite-bed friction angles were measured as described in the procedure given above. The composite-bed friction angle of the swollen composite were found to be 25 degrees, 25 degrees, 24 degrees, and 23 degrees, respectively.

### Example 2

An air-formed composite was made from 40% weight (on dry basis) superabsorbent material, untreated FAVOR® SXM 9543 (from Control 1) and 60% weight (on dry basis) fluff fiber of NB416 (from Control 1) blended with T255 (from Example 1) at a ratio of 0.5 grams NB416 and 0.5 grams of T255 per 1.0 grams of fiber. The composite was swollen, following the method given above, in solutions of 20%, 10%, 5% and 0.9% by weight aqueous NaCl solution. The composite-bed friction angles were measured as described in the procedure given above. The composite-bed friction angle of the swollen composite were found to be 26 degrees, 23 degrees, 24 degrees, and 16 degrees, respectively.

### Example 3

An air-formed composite was made from 60% weight (on dry basis) superabsorbent material, untreated FAVOR® SXM 9543 (from Control 1) and 40% weight (on dry basis) fluff fiber of NB416 (from Control 1) blended with T255 (from Example 1) at a ratio of 0.5 grams NB416 and 0.5 grams of T255 per 1.0 grams of fiber. The composite was swollen, following the method given above, in solutions of 20%, 10%, 5% and 0.9% by weight aqueous NaCl solution. The composite-bed friction angles were measured as described in the procedure given above. The composite-bed friction angle of the swollen composite were found to be 25 degrees, 24 degrees, 23 degrees, and 18 degrees, respectively.

### Example 4

An air-formed composite was made from 20% weight (on dry basis) superabsorbent material, untreated FAVOR® SXM 9543 (from Control 1) and 80% weight (on dry basis) fluff fiber of NB416 (from Control 1) blended with T255, a synthetic KoSa Celbond® bicomponent fiber available from KoSa, at a ratio of 0.5 grams NB416 and 0.5 grams of T255 per 1.0 grams of fiber. The composite was swollen, following the method given above, in solutions of 20%, 10%, 5% and 0.9% by weight aqueous NaCl solution. The composite-bed cohesion values were measured as described in the procedure given above. The composite-bed cohesion value of the swollen composite were found to be 1104 Pascals, 1140 Pascals, 1034 Pascals, and 1099 Pascals, respectively.

### Example 5

An air-formed composite was made from 40% weight (on dry basis) superabsorbent material, untreated FAVOR® SXM 9543 (from Control 1) and 60% weight (on dry basis) fluff fiber of NB416 (from Control 1) blended with T255 (from Example 1) at a ratio of 0.5 grams NB416 and 0.5 grams of T255 per 1.0 grams of fiber. The composite was swollen, following the method given above, in solutions of 20%, 10%, 5% and 0.9% by weight aqueous NaCl solution. The composite-bed cohesion values were measured as described in the procedure given above. The composite-bed cohesion value of the swollen composite were found to be 1036 Pascals, 1182 Pascals, 1188 Pascals, and 907 Pascals, respectively.

### Example 6

An air-formed composite was made from 60% weight (on dry basis) superabsorbent material, untreated FAVOR® SXM 9543 (from Control 1) and 40% weight (on dry basis) fluff fiber of NB416 (from Control 1) blended with T255 (from Example 1) at a ratio of 0.5 grams NB416 and 0.5 grams of T255 per 1.0 grams of fiber. The composite was swollen, following the method given above, in solutions of 20%, 10%, 5% and 0.9% by weight aqueous NaCl solution. The composite-bed cohesion values were measured as described in the procedure given above. The composite-bed cohesion value of the swollen composite were found to be 1137 Pascals, 1142 Pascals, 1336 Pascals, and 856 Pascals, respectively.

### Example 7

An air-formed composite was made from 40% weight (on dry basis) superabsorbent material, untreated FAVOR® SXM 9543 (from Control 1) and 60% weight (on dry basis) fluff fiber of NB416 (from Control 1) coated with Mineral Oil, CAS 8012-95-1, available from Mallinckrodt Baker, having business offices in Phillipsburg, N.J., and Lecithin, CAS 8002-43-5, available from Spectrum Quality Products, Inc., a business having offices in Gardena, Calif., in a ratio of 0.2 grams of additive per 1.0 grams of fiber. The coating/additive was a mixture containing 0.95 grams of mineral oil and 0.05 grams of Lecithin for every 1.0 gram of additive. The composite was swollen, following the method given above, in solutions of 20%, 10%, and 0.9% by weight aqueous NaCl solution. The composite-bed cohesion values were measured as described in the procedure given above. The composite-bed cohesion value of the swollen composite were found to be 1098 Pascals, 1150 Pascals, and 1325 Pascals, respectively.

While the embodiments of the present invention described herein are presently preferred, various modifications and improvements may be made without departing from the spirit and scope of the present invention. The scope of the present invention is indicated by the appended claims, and all changes that fall within the meaning and range of equivalents are intended to be embraced therein.

We claim:

1. An absorbent composite, comprising:
a fibrous matrix; and, a water swellable, water insoluble superabsorbent material in combination with the fibrous matrix in a dosage level of between about 20 and about 40 percent, the absorbent composite having a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals, substantially equal to or less than the first composite-bed friction angle,
wherein the first composite-bed friction angle is about 30 degrees or less.

2. The absorbent composite of claim 1, wherein in the first composite-bed friction angle is about 20 degrees or less.

3. The absorbent composite of claim 1, wherein the absorbent composite has a composite-bed cohesion value of about 10,000 Pascals or less when swollen in a NaCl solution having a NaCl concentration of about 20% by weight for one hour under an external load of 2,000 Pascals.

4. The absorbent composite of claim 1, wherein the water swellable, water insoluble superabsorbent material is selected from the group consisting essentially of natural materials, synthetic materials, modified natural materials, and combinations thereof.

5. The absorbent composite of claim 4, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof.

6. The absorbent composite of claim 1, wherein the water swellable, water insoluble superabsorbent material further comprises a structure selected from the group consisting essentially of particles, fibers, flakes, spheres, and combinations thereof.

7. An absorbent composite, comprising:
a fibrous matrix; and,
a water swellable, water insoluble superabsorbent material in combination with the fibrous matrix in a dosage level of between about 40 and about 60 percent, the absorbent composite having a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals, substantially equal to or less than the first composite-bed friction angle,
wherein the first composite-bed friction angle is about 27 degrees or less.

8. The absorbent composite of claim 7, wherein in the first composite-bed friction angle is about 17 degrees or less.

9. The absorbent composite of claim 7, wherein the absorbent composite has a composite-bed cohesion value of about 10,000 Pascals or less when swollen in a NaCl solution having a NaCl concentration of about 20% by weight for one hour under an external load of 2,000 Pascals.

10. The absorbent composite of claim 7, wherein the water swellable, water insoluble superabsorbent material is selected from the group consisting essentially of natural materials, synthetic materials, modified natural materials, and combinations thereof.

11. The absorbent composite of claim 10, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof.

12. The absorbent composite of claim 7, wherein the water swellable, water insoluble superabsorbent material further comprises a structure selected from the group consisting essentially of particles, fibers, flakes, spheres, and combinations thereof.

13. An absorbent composite, comprising:
a fibrous matrix; and,
a water swellable, water insoluble superabsorbent material in combination with the fibrous matrix in a dosage level of between about 60 and about 80 percent, the absorbent composite having a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals, substantially equal to or less than the first composite-bed friction angle,
wherein the first composite-bed friction angle is about 25 degrees or less.

14. The absorbent composite of claim 13, wherein in the first composite-bed friction angle is about 15 degrees or less.

15. The absorbent composite of claim 13, wherein the absorbent composite has a composite-bed cohesion value of about 10,000 Pascals or less when swollen in a NaCl solution having a NaCl concentration of about 20% by weight for one hour under an external load of 2,000 Pascals.

16. The absorbent composite of claim 13, wherein the water swellable, water insoluble superabsorbent material is selected from the group consisting essentially of natural materials, synthetic materials, modified natural materials, and combinations thereof.

17. The absorbent composite of claim 16, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof.

18. The absorbent composite of claim 13, wherein the water swellable, water insoluble superabsorbent material further comprises a structure selected from the group consisting essentially of particles, fibers, flakes, spheres, and combinations thereof.

19. An absorbent composite, comprising:
a fibrous matrix; and,
a water swellable, water insoluble superabsorbent material in combination with the fibrous matrix in a dosage level of between about 20 and about 80 percent, the absorbent composite having a composite-bed cohesion value when swollen in a NaCl solution having a NaCl concentration of about 20% by weight for one hour under an external load of 2,000 Pascals of about 1,200 Pascals or less.

20. The absorbent composite of claim 19, wherein in the composite-bed cohesion value is about 500 Pascals or less.

21. The absorbent composite of claim 19, wherein the water swellable, water insoluble superabsorbent material is selected from the group consisting essentially of natural materials, synthetic materials, modified natural materials, and combinations thereof.

22. The absorbent composite of claim 21, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof.

23. The absorbent composite of claim 19, wherein the water swellable, water insoluble superabsorbent material further comprises a structure selected from the group consisting essentially of particles, fibers, flakes, spheres, and combinations thereof.

24. An absorbent composite, comprising:
a fibrous matrix; and,
a water swellable, water insoluble superabsorbent material in combination with the fibrous matrix in a dosage level of between about 20 and about 40 percent, the absorbent composite having a first composite-bed friction angle when swollen in a NaCl solution having a NaCl concentration of about 5% by weight for one hour under an external load of 2,000 Pascals and composite-bed friction angles, when swollen in a NaCl solution having a NaCl concentration of less than about 5% by weight for one hour under an external load of 2,000 Pascals, substantially equal to or greater than the first composite-bed friction angle,
wherein the first composite-bed friction angle is about 39 degrees or greater.

25. The absorbent composite of claim 24, wherein in the first composite-bed friction angle is about 46 degrees or greater.

26. The absorbent composite of claim 24, wherein the absorbent composite has a composite-bed cohesion value of about 100 Pascals or greater when swollen in a NaCl solution having a NaCl concentration of about 5% by weight for one hour under an external load of 2,000 Pascals.

27. The absorbent composite of claim 24, wherein the water swellable, water insoluble superabsorbent material is selected from the group consisting essentially of natural materials, synthetic materials, modified natural materials, and combinations thereof.

28. The absorbent composite of claim 27, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof.

29. The absorbent composite of claim 27, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, and combinations thereof.

30. The absorbent composite of claim 24, wherein the water swellable, water insoluble superabsorbent material further comprises a structure selected from the group consisting essentially of particles, fibers, flakes, spheres, and combinations thereof.

31. An absorbent composite, comprising:
a fibrous matrix; and,
a water swellable, water insoluble superabsorbent material in combination with the fibrous matrix in a dosage level of between about 40 and about 60 percent, the absorbent composite having a first composite-bed friction angle when swollen in a NaCl solution having a NaCl concentration of about 5% by weight for one hour under an external load of 2,000 Pascals and composite-bed friction angles, when swollen in a NaCl solution having a NaCl concentration of less than about 5% by weight for one hour under an external load of 2,000 Pascals, substantially equal to or greater than the first composite-bed friction angle,
wherein the first composite-bed friction angle is about 35 degrees or greater.

32. The absorbent composite of claim 31, wherein in the first composite-bed friction angle is about 42 degrees or greater.

33. The absorbent composite of claim 31, wherein the absorbent composite has a composite-bed cohesion value of about 100 Pascals or greater when swollen in a NaCl solution having a NaCl concentration of about 5% by weight for one hour under an external load of 2,000 Pascals.

34. The absorbent composite of claim 31, wherein the water swellable, water insoluble superabsorbent material is selected from the group consisting essentially of natural materials, synthetic materials, modified natural materials, and combinations thereof.

35. The absorbent composite of claim 34, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof.

36. The absorbent composite of claim 34, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, and combinations thereof.

37. The absorbent composite of claim 31, wherein the water swellable, water insoluble superabsorbent material further comprises a structure selected from the group consisting essentially of particles, fibers, flakes, spheres, and combinations thereof.

38. An absorbent composite, comprising:
a fibrous matrix; and,
a water swellable, water insoluble superabsorbent material in combination with the fibrous matrix in a dosage level of between about 60 and about 80 percent, the absorbent composite having a first composite-bed friction angle when swollen in a NaCl solution having a NaCl concentration of about 5% by weight for one hour under an external load of 2,000 Pascals and composite-bed friction angles, when swollen in a NaCl solution having a NaCl concentration of less than about 5% by weight for one hour under an external load of 2,000 Pascals, substantially equal to or greater than the first composite-bed friction angle,
wherein the first composite-bed friction angle is about 33 degrees or greater.

39. The absorbent composite of claim 38, wherein in the first composite-bed friction angle is about 40 degrees or greater.

40. The absorbent composite of claim 38, wherein the absorbent composite has a composite-bed cohesion value of about 100 Pascals or greater when swollen in a NaCl solution having a NaCl concentration of about 5% by weight for one hour under an external load of 2,000 Pascals.

41. The absorbent composite of claim 38, wherein the water swellable, water insoluble superabsorbent material is selected from the group consisting essentially of natural materials, synthetic materials, modified natural materials, and combinations thereof.

42. The absorbent composite of claim 41, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof.

43. The absorbent composite of claim 41, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, and combinations thereof.

44. The absorbent composite of claim 38, wherein the water swellable, water insoluble superabsorbent material further comprises a structure selected from the group consisting essentially of particles, fibers, flakes, spheres, and combinations thereof.

45. An absorbent composite, comprising:
a fibrous matrix; and,
a water swellable, water insoluble superabsorbent material in combination with the fibrous matrix in a dosage level of between about 20 and about 80 percent, the absorbent composite having a composite-bed cohesion value when swollen in a NaCl solution having a NaCl concentration of about 20% by weight for one hour under an external load of 2,000 Pascals of about 4,500 Pascals or greater.

46. The absorbent composite of claim 45, wherein in the composite-bed cohesion value is about 6,500 Pascals or greater.

47. The absorbent composite of claim 45, wherein the water swellable, water insoluble superabsorbent material is selected from the group consisting essentially of natural materials, synthetic materials, modified natural materials, and combinations thereof.

48. The absorbent composite of claim 47, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof.

49. The absorbent composite of claim 45, wherein the water swellable, water insoluble superabsorbent material further comprises a structure selected from the group consisting essentially of particles, fibers, flakes, spheres, and combinations thereof.

50. An absorbent composite, comprising:
a fibrous matrix; and,
a water swellable, water insoluble superabsorbent material in combination with the fibrous matrix in a dosage level of between about 20 and about 80 percent, the absorbent composite having a composite-bed cohesion value when swollen in a NaCl solution having a NaCl concentration of about 5% by weight for one hour under an external load of 2,000 Pascals of about 3,000 Pascals or greater.

51. The absorbent composite of claim 50, wherein in the composite-bed cohesion value is about 5,000 Pascals or greater.

52. The absorbent composite of claim 50, wherein the water swellable, water insoluble superabsorbent material is selected from the group consisting essentially of natural materials, synthetic materials, modified natural materials, and combinations thereof.

53. The absorbent composite of claim 52, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof.

54. The absorbent composite of claim 52, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, and combinations thereof.

55. The absorbent composite of claim 50, wherein the water swellable, water insoluble superabsorbent material further comprises a structure selected from the group consisting essentially of particles, fibers, flakes, spheres, and combinations thereof.

56. An absorbent composite, comprising:
a fibrous matrix; and,
a water swellable, water insoluble superabsorbent material in combination with the fibrous matrix in a dosage level of between about 20 and about 40 percent, the absorbent composite having a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals, substantially equal to or greater than the first composite-bed friction angle,
wherein the first composite-bed friction angle is about 30 degrees or less.

57. The absorbent composite of claim 56, wherein in the first composite-bed friction angle is about 20 degrees or less.

58. The absorbent composite of claim 56, wherein the absorbent composite has a composite-bed cohesion value of about 10,000 Pascals or less when swollen in a NaCl solution having a NaCl concentration of about 20% by weight for one hour under an external load of 2,000 Pascals.

59. The absorbent composite of claim 56, wherein the water swellable, water insoluble superabsorbent material is selected from the group consisting essentially of natural materials, synthetic materials, modified natural materials, and combinations thereof.

60. The absorbent composite of claim 59, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof.

61. The absorbent composite of claim 56, wherein the water swellable, water insoluble superabsorbent material further comprises a structure selected from the group consisting essentially of particles, fibers, flakes, spheres, and combinations thereof.

62. An absorbent composite, comprising:
a fibrous matrix; and,
a water swellable, water insoluble superabsorbent material in combination with the fibrous matrix in a dosage level of between about 40 and about 60 percent, the absorbent composite having a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals, substantially equal to or greater than the first composite-bed friction angle,
wherein the first composite-bed friction angle is about 27 degrees or less.

63. The absorbent composite of claim 62, wherein in the first composite-bed friction angle is about 17 degrees or less.

64. The absorbent composite of claim 62, wherein the absorbent composite has a composite-bed cohesion value of about 10,000 Pascals or less when swollen in a NaCl solution having a NaCl concentration of about 20% by weight for one hour under an external load of 2,000 Pascals.

65. The absorbent composite of claim 62, wherein the water swellable, water insoluble superabsorbent material is selected from the group consisting essentially of natural materials, synthetic materials, modified natural materials, and combinations thereof.

66. The absorbent composite of claim 65, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof.

67. The absorbent composite of claim 62, wherein the water swellable, water insoluble superabsorbent material further comprises a structure selected from the group consisting essentially of particles, fibers, flakes, spheres, and combinations thereof.

68. An absorbent composite, comprising:
a fibrous matrix; and,
a water swellable, water insoluble superabsorbent material in combination with the fibrous matrix in a dosage level of between about 60 and about 80 percent, the absorbent composite having a first composite-bed friction angle when swollen in a NaCl solution having a NaCl concentration of about 20% by weight for one hour under an external load of 2,000 Pascals and composite-bed friction angles, when swollen in a NaCl solution having a NaCl concentration of less than about 20% by weight for one hour under an external load of 2,000 Pascals, substantially equal to or greater than the first composite-bed friction angle,
wherein the first composite-bed friction angle is about 25 degrees or less.

69. The absorbent composite of claim 68, wherein in the first composite-bed friction angle is about 15 degrees or less.

70. The absorbent composite of claim 68, wherein the absorbent composite has a composite-bed cohesion value of about 10,000 Pascals or less when swollen in a NaCl solution having a NaCl concentration of about 20% by weight for one hour under an external load of 2,000 Pascals.

71. The absorbent composite of claim 68, wherein the water swellable, water insoluble superabsorbent material is selected from the group consisting essentially of natural materials, synthetic materials, modified natural materials, and combinations thereof.

72. The absorbent composite of claim 71, wherein the water swellable, water insoluble superabsorbent material is selected from the group consisting essentially of silica gels, agar, pectin, guar gum, alkali metal salts of polyacrylic acids, polyacrylamides, polyvinyl alcohols, ethylene maleic anhydride copolymers, polyvinyl ethers, hydroxypropylcelluloses, polyvinyl morpholinones, polymers and copolymers of vinyl sulfonic acid, polyacrylates, polyacrylamides, polyvinyl pyridine, acrylonitrile grafted starch, acrylic acid grafted starch, isobutylene maleic anhydride copolymers, polyamines, and combinations thereof.

73. The absorbent composite of claim 68, wherein the water swellable, water insoluble superabsorbent material further comprises a structure selected from the group consisting essentially of particles, fibers, flakes, spheres, and combinations thereof.
